(19) United States
(12) Patent Application Publication
(10) Pub. No.: US 2021/0308238 A1
(43) Pub. Date: Oct. 7, 2021

(54) INJECTION SOLUTION FOR RNA

(71) Applicant: CureVac AG, Tubingen (DE)

(72) Inventors: Ingmar HOERR, Tübingen (DE); Steve PASCOLO, Tübingen (DE)

(73) Assignee: CureVac AG, Tübingen (DE)

(21) Appl. No.: 17/200,693

(22) Filed: Mar. 12, 2021

Related U.S. Application Data
(63) Continuation of application No. 15/835,403, filed on Dec. 7, 2017, now abandoned.
Application No. 15/835,403 is a continuation of application No. 11/914,945, filed on Jul. 7, 2008, now abandoned.

(30) Foreign Application Priority Data
May 19, 2005 (DE) 10 2005 023 170.5

(51) Int. Cl. A61K 39/00; A61K 9/00; A61K 31/7088; A61K 31/7105; A61K 47/02; A61K 47/12; A61K 48/00

(52) U.S. Cl. CPC A61K 39/00; A61K 9/0019; A61K 31/7088; A61K 31/7105; A61K 2039/53; A61K 47/12; A61K 48/0008; A61K 48/005; A61K 47/02

(57) ABSTRACT

The invention relates to the use of RNA and an aqueous injection buffer containing a sodium salt, a calcium salt and optionally a potassium salt and optionally lactate, in the preparation of a RNA injection solution for increasing RNA transfer and/or RNA translation into/in a host organism. The invention relates further to a RNA injection solution and to a method for increasing the RNA transfer and/or RNA translation of RNA in vivo and in vitro.

This application is a continuation of U.S. application Ser. No. 15/835,403, filed Dec. 7, 2017, which is a continuation of U.S. application Ser. No. 11/914,945, filed Jul. 7, 2008, now abandoned, which is a national phase application under 35 U.S.C. § 371 of International Application No. PCT/EP2006/004784, filed May 19, 2006, which claims benefit of German Application No. 102005023170.5, filed May 19, 2005, the entire contents of each of which are hereby incorporated by reference.

The invention relates to the use of RNA and an aqueous injection buffer containing a sodium salt, a calcium salt, optionally a potassium salt and optionally also lactate, in the preparation of a RNA injection solution for increasing RNA transfer and/or RNA translation into/in a host organism.

Molecular-medical processes, such as gene therapy and genetic vaccination, play a large part in the therapy and prevention of numerous diseases. Such processes are based on the introduction of nucleic acids into the patient's cells or tissue, followed by processing of the information coded for by the nucleic acids that have been introduced, that is to say translation into the desired polypeptides or proteins. Both DNA and RNA come into consideration as nucleic acids that can be introduced.

Genetic vaccinations, which consist in the injection of naked plasmid DNA, were demonstrated for the first time in the early 1990s on mice. However, it became clear during clinical phase I/II studies that, in humans, this technology was unable to fulfil the expectations awakened by the studies in mice¹. Numerous DNA-based genetic vaccinations and methods for introducing DNA into cells (inter alia calcium phosphate transfection, polyprene transfection, protoplast fusion, electroporation, microinjection, lipofection, use of DNA viruses as DNA vehicles) have since been developed.

15 years ago, Wolff et al. showed that the injection of naked genetic information in the form of plasmid DNA (p-DNA) or mRNA in mice can lead to protein expression². These results were followed by investigations which showed that naked pDNA can be used for a vaccination³⁻⁵. The use of mRNA for vaccination, however, was paid little attention until the late 1990s, when it was demonstrated that the transfer of mRNA into dendritic cells triggers immune responses⁶. The direct injection of naked mRNA for vaccination remained a marginal theme, however, and was discussed in only four articles by three different working groups⁷⁻¹⁰. One of the mains reasons for this was the instability of mRNA owing to its rapid decomposition by ribonucleases and the associated limited effectiveness of the mRNA as a genetic tool in vivo. In the meantime, however, numerous methods for stabilising mRNA have been described in the prior art, for example in EP-A-1083232, WO 99/14346, U.S. Pat. Nos. 5,580,859 and 6,214,804.

RNA as the nucleic acid for a genetic vehicle has numerous advantages over DNA, including:
i) the RNA introduced into the cell does not integrate into the genome (whereas DNA does integrate into the genome to a certain degree and can thereby insert into an intact gene of the genome of the host cell, so that this gene is able to mutate and can lead to a partial or total loss of the genetic information or to misinformation),
ii) no viral sequences, such as promoters, etc., are required for the effective transcription of RNA (whereas a strong promoter (e.g. the viral CMV promoter) is required for the expression of DNA introduced into the cell). The integration of such promoters into the genome of the host cell can lead to undesirable changes in the regulation of gene expression),
iii) the degradation of RNA that has been introduced takes place in a limited time (several hours)^{11, 12}, so that it is possible to achieve transient gene expression which can be discontinued after the required treatment period (whereas this is not possible in the case of DNA that has been integrated into the genome),
iv) RNA does not lead to the induction of pathogenic anti-RNA antibodies in the patient (whereas the induction of anti-DNA antibodies is known to cause an undesirable immune response),
v) RNA is widely usable—any desired RNA for any desired protein of interest can be prepared at short notice for a vaccination, even on an individual patient basis.

In summary, it remains to be noted that mRNA represents a transient copy of the coded genetic information in all organisms, serves as a model for the synthesis of proteins and, unlike DNA, represents all the necessary prerequisites for the preparation of a suitable vector for the transfer of exogenous genetic information in vivo.

A particularly suitable procedure for the described transfer of nucleic acids into a host organism, in particular a mammal, is the injection thereof. While DNA for such injections is conventionally diluted in water, NaCl or PBS injection buffer, RNA is conventionally diluted only in an injection buffer. There are used as RNA injection buffers standard buffers, such as phosphate-buffered salt solutions, in particular PBS and HEPES-buffered salt solution (HBS). In the case of the transfer of mRNA, such a RNA injection solution is preferably heated for a short time prior to its administration in order to remove secondary structures of the mRNA. A disadvantage when using such standard buffers for RNA injection solutions is that the intradermal transfer of the RNA is only very inefficient. A further disadvantage is that the translation rate of the transferred RNA is very low. A further disadvantage is that the RNA frequently forms a secondary structure (e.g. a so-called hairpin structure) in such standard buffers, which can greatly reduce the effectiveness of the uptake of the RNA into the cytosol.

The object of the present invention is, therefore, to provide a system with which on the one hand intradermal RNA transfer into a host organism is improved and on the other hand the translation rate of the transferred RNA is increased.

This object is achieved by the embodiments of the invention characterised in the claims.

The present invention provides the use of RNA and an aqueous injection buffer containing a sodium salt, preferably at least 50 mM of a sodium salt, a calcium salt, preferably at least 0.01 mM of a calcium salt, and optionally a potassium salt, preferably at least 3 mM of a potassium salt, in the preparation of a RNA injection solution for increasing RNA transfer and/or RNA translation into/in a host organism. A further aspect of the present invention also provides an injection solution so prepared. The injection solution is obtained, therefore, from the injection buffer and the RNA dissolved in the injection buffer.

According to a preferred embodiment, the sodium salts, calcium salts and optionally potassium salts contained in the injection buffer are in the form of halides, for example chlorides, iodides or bromides, in the form of their hydroxides, carbonates, hydrogen carbonates or sulfates. Examples which may be mentioned here are: for the sodium salt, NaCl, NaI, NaBr, Na₂CO₃, NaHCO₃, Na₂SO₄; for the potassium salt which is optionally present, KCl, KI, KBr, K₂CO₃, KHCO₃, K₂SO₄, and for the calcium salt, CaCl₂, CaI₂, CaBr₂, CaCO₃, CaSO₄, Ca(OH)₂. Organic ions of the above-mentioned cations can also be contained in the injection buffer.

In a particularly preferred embodiment of the use according to the invention of RNA and an injection buffer, an injection buffer according to the invention contains as salts sodium chloride (NaCl), calcium chloride (CaCl₂) and optionally potassium chloride (KCl), it being possible for other anions also to be present in addition to the chlorides. These salts are typically present in the injection buffer in a concentration of at least 50 mM sodium chloride (NaCl), at least 3 mM potassium chloride (KCl) and at least 0.01 mM calcium chloride (CaCl₂).

The injection buffer according to the invention can be present both as a hypertonic or an isotonic or hypotonic injection buffer. In connection with the present invention, the injection buffer is hypertonic, isotonic or hypotonic in relation to the respective reference medium, that is to say the injection buffer according to the invention has a higher, equal or lower salt content as compared with the respective reference medium, the concentrations of the above-mentioned salts that are used preferably being those which do not result in damage to the cells caused by osmosis or other concentration effects. Reference media here are, for example, liquids that occur in “in vivo” processes, such as, for example, blood, lymph fluid, cytosolic fluids or other fluids that occur in the body, or liquids or buffers conventionally used in “in vitro” processes. Such liquids and buffers are known to a person skilled in the art.

The injection buffer can contain further components, for example sugars (mono-, di-, tri- or poly-saccharides), in particular glucose or mannitol. In a preferred embodiment, however, no sugars will be present in the injection buffer employed for the use according to the invention. It is also preferable for the buffer according to the invention not to contain any uncharged components, such as, for example, sugars. The buffer according to the invention typically contains only metal cations, in particular from the group of the alkali or alkaline earth metals, and anions, in particular the anions mentioned above.

The pH value of the injection buffer of the present invention is preferably from 1 to 8.5, preferably from 3 to 5, more preferably from 5.5 to 7.5, especially from 5.5 to 6.5. The injection buffer can optionally also contain a buffer system, which fixes the injection buffer at a buffered pH value. Such a system can be, for example, a phosphate buffer system, HEPES or Na₂HPO₄/NaH₂PO₄. However, very particular preference is given to the injection buffer used according to the invention when it does not contain any of the above-mentioned buffer systems or no buffer system at all.

The injection buffer used according to the invention contains, as described hereinbefore, salts of sodium, calcium and optionally potassium, sodium and calcium typically being present in the injection buffer in the form of monovalent cations (Na⁺, K⁺) and calcium being present in the form of the divalent cation (Ca²⁺). According to a preferred embodiment, in addition to these or alternatively thereto, monovalent and divalent cations contained in the injection buffer used according to the invention can be divalent cations, in particular from the group of the alkaline earth metals, such as, for example, magnesium (Mg²⁺), or also iron (Fe²⁺), and monovalent cations, in particular from the group of the alkali metals, such as, for example, lithium (Lit). These monovalent cations are preferably present in the form of their salts, for example in the form of halides, e.g. chlorides, iodides or bromides, in the form of their hydroxides, carbonates, hydrogen carbonates or sulfates. Examples which may be mentioned here are: for the lithium salt, LiCl, LiI, LiBr, Li₂CO₃, LiHCO₃, Li₂SO₄; for the magnesium salt, MgCl₂, MgI₂, MgBr₂, MgCO₃, MgSO₄ and Mg(OH)₂; and for the iron salt, FeCl₂, FeBr₂, FeI₂, FeF₂, Fe₂O₃, FeCO₃, FeSO₄, Fe(OH)₂. Also included are all combinations of divalent and/or monovalent cations, as described hereinbefore. Thus, injection buffers according to the invention that contain only divalent, only monovalent or divalent and monovalent cations are included. Also included are injection buffers according to the invention that contain only one type of divalent or monovalent cations, particularly preferably, for example, only Ca²⁺ cations or a salt thereof, for example CaCl₂.

It is preferable for the molarities indicated above for Ca²⁺ (as divalent cation) and Na¹⁺ (as monovalent cation) (that is to say typically concentrations of at least 50 mM Nat, at least 0.01 mM Ca²⁺ and optionally at least 3 mM K⁺) also to be taken into consideration in the injection buffer when, instead of some or all of the Ca²⁺ or Na¹⁺, a different divalent or monovalent cation or different divalent or monovalent cations, in particular different cations from the group of the alkaline earth metals or alkali metals, are employed in the injection buffer used according to the invention for the preparation of the injection solution. Although Ca²⁺ and Na¹⁺, as mentioned above, can be replaced completely by different divalent and monovalent cations in the injection buffer used according to the invention, for example also by a combination of different divalent cations (instead of Ca²⁺) and/or a combination of different monovalent cations (instead of Na¹⁺) (in particular a combination of different divalent cations from the group of the alkaline earth metals or a combination of different monovalent cations from the group of the alkali metals), it is preferred to replace Ca²⁺ or Na¹⁺ partially, that is to say to fill at least 20%, preferably at least 40%, more preferably at least 60% and yet more preferably at least 80%, of the respective total molarities of the monovalent or divalent cations in the injection buffer with Ca²⁺ or Na¹⁺. However, it is very particularly preferred for the injection buffer used according to the invention to contain only Ca²⁺ as divalent cation and Na¹⁺ as monovalent cation, that is to say Ca²⁺ represents 100% of the total molarity of divalent cations and Na¹⁺ represents 100% of the total molarity of monovalent cations.

The preparation of the injection buffer is preferably carried out at room temperature (25° C.) and atmospheric pressure. The preparation can be carried out according to any desired process from the prior art. Preferably, the ions or salts contained therein are diluted in aqueous solution, whereby the concentration ratios are to be chosen according to the particular conditions (host organism, in particular mammal, into which the RNA injection solution is injected, state of health, age, etc. of the host organism, and conditions of solubility and interference of the components, reaction temperature, reaction time, etc.).

The concentrations of the components sodium, calcium and chloride ions and optionally potassium ions and optionally lactate (see the embodiments hereinbelow) contained in the aqueous injection buffer are dependent in particular on their solubility in water, the interference of the components with one another, as well as on the reaction temperature and reaction pressure during the preparation of the injection buffer or of the RNA injection solution.

The injection buffer used according to the present invention is based on an aqueous solution, that is to say on a solution consisting of water and the salts used according to the invention for the injection solution, and optionally lactate. The salts of the above-mentioned monovalent or divalent cations can optionally be sparingly soluble or even insoluble in such an aqueous solution. The degree of solubility of the salts can be calculated from the solubility product. Processes for the precise determination of the solubility and of the solubility product are known to a person skilled in the art. This aqueous solution can contain up to 30 mol % of the salts contained in the solution, especially up to 25 mol %, preferably up to 20 mol %, also preferably up to 15 mol %, more preferably up to 10 mol %, yet more preferably up to 5 mol %, likewise more preferably up to 2 mol %, insoluble or sparingly soluble salts. Salts whose solubility product is <10⁻⁴ are considered to be sparingly soluble within the scope of the present invention. Salts whose solubility product is >10⁻⁴ are considered to be readily soluble.

The solubility of a salt or of an ion or ion compound in water depends on its lattice energy and the hydration energy, taking into account entropy effects that occur. The term solubility product is also used, more precisely the equilibrium that is established when a salt or an ion or ion compound dissolves in water. The solubility product is generally defined as the product of the concentrations of the ions in the saturated solution of an electrolyte. For example, alkali metals (such as, for example, Na⁺, K⁺) are soluble in water in higher concentrations than alkaline earth metal salts (such as, for example, Ca²⁺ salts), that is to say they have a greater solubility product. That is to say, the potassium and sodium salts contained in the aqueous solution of the injection buffer according to the invention are more readily soluble than the calcium salts that are present. Therefore, it is necessary when determining the concentration of these ions to take into consideration, inter alia, the interference between the potassium, sodium and calcium salts.

Preference is given to a use according to the invention in which the injection buffer contains from 50 mM to 800 mM, preferably from 60 mM to 500 mM, more preferably from 70 mM to 250 mM, particularly preferably from 60 mM to 110 mM, sodium chloride (NaCl), from 0.01 mM to 100 mM, preferably from 0.5 mM to 80 mM, more preferably from 1.5 mM to 40 mM, calcium chloride (CaCl₂) and optionally from 3 mM to 500 mM, preferably from 4 mM to 300 mM, more preferably from 5 mM to 200 mM, potassium chloride (KCl).

In addition to the above-mentioned inorganic anions, for example halides, sulfates or carbonates, organic anions can also occur as further anions. Among these, mention may be made of succinate, lactobionate, lactate, malate, maleonate, etc., which can also be present in combinations. An injection buffer for use according to the invention preferably contains lactate, particularly preferably such an injection buffer, where an organic anion is present, contains only lactate as organic anion. Lactate within the scope of the invention can be any desired lactate, for example L-lactate and D-lactate. In connection with the present invention, sodium lactate and/or calcium lactate typically occur as lactate salts, in particular when the injection buffer contains only Na as monovalent cation and Ca²⁺ as divalent cation.

In a preferred form of the use according to the invention, an injection buffer according to the invention contains preferably from 15 mM to 500 mM, more preferably from 15 mM to 200 mM and yet more preferably most preferably from 15 mM to 100 mM, lactate.

It has been found according to the invention that the use of an injection buffer having the components described above, optionally with or without lactate (hereinbelow: “RL injection buffer” when the component lactate is not present, or “RL injection buffer with lactate” when the component lactate is present), for RNA injection solutions (i.e. injection solutions which contain RNA and are suitable for the injection of that RNA) significantly increases both the transfer and the translation of the RNA in/into the cells/tissue of a host organism (mammal) as compared with the injection buffers conventionally used in the prior art.

A solution having the above-mentioned components sodium chloride (NaCl), calcium chloride (CaCl₂)), lactate, in particular sodium lactate, and optionally also potassium chloride (KCl) is also known as “Ringer's solution” or “Ringer's lactate”. Ringer's lactate is a crystalloid full electrolyte solution which is used as a volume replacement and as a carrier solution, for example for compatible medicaments. For example, Ringer's lactate is used as a primary volume replacement agent in cases of fluid and electrolyte loss (through vomiting, diarrhea, intestinal obstruction or burns), in particular in infants and small children, and for keeping open peripheral and/or central venous accesses. The use according to the invention of Ringer's lactate as an injection buffer in a RNA injection solution is not described in the prior art, however.

RNA within the scope of the invention is any desired RNA, for example mRNA, tRNA, rRNA, siRNA, single- or double-stranded RNA, heteroduplex RNA, etc. The RNA used can code for any protein that is of interest. The RNA used according to the invention is preferably naked RNA. Particularly preferably, it is mRNA, more preferably naked mRNA.

Naked RNA, in particular naked mRNA, within the scope of the invention is to be understood as being a RNA that is not complexed, for example with polycationic molecules. Naked RNA can be present in single-stranded form but also in double-stranded form, that is to say as a secondary structure, for example as a so-called “hairpin structure”. Such double-stranded forms occur especially within the naked RNA, in particular the naked mRNA, when complementary ribonucleotide sequences are present in the molecule.

According to the invention, however, the RNA, in particular mRNA, can also be present in complexed form. As a result of such complexing/condensation of the RNA, in particular mRNA, of the invention, the effective transfer of the RNA into the cells that are to be treated or into the tissue that is to be treated of the organism to be treated can be improved by associating or binding the RNA with a (poly)cationic polymer, peptide or protein. Such a RNA (mRNA) is preferably complexed or condensed with at least one cationic or polycationic agent. Such a cationic or polycationic agent is preferably an agent selected from the group consisting of protamine, poly-L-lysine, poly-L-arginine, nucleolin, spermine and histones or derivatives of histones or protamines. Particular preference is given to the use of protamine as polycationic, nucleic-acid-binding protein. This procedure for stabilising the RNA is described, for example, in EP-A-1083232, the relevant disclosure of which is incorporated in its entirety into the present invention.

The RNA of the invention can further be modified. These modifications serve especially to increase the stability of the RNA. The RNA preferably has one or more (naturally occurring or non-natural) modifications, in particular chemical modifications, which, for example, contribute to increasing the half-life of the RNA in the organism or improve the translation efficiency of the mRNA in the cytosol as compared with the translation efficiency of unmodified mRNA in the cytosol. Preferably, the translation efficiency is improved by a modification according to the invention by at least 10%, preferably at least 20%, likewise preferably by at least 40%, more preferably by at least 50%, yet more preferably by at least 60%, likewise more preferably by at least 75%, most preferably by at least 85%, most preferably by at least 100%, as compared with the translation efficiency of unmodified mRNA in the cytosol.

For example, the G/C content of the coding region of a modified mRNA can be increased as compared with the G/C content of the coding region of the corresponding wild-type mRNA, the coded amino acid sequence of the modified mRNA preferably remaining unchanged relative to the coded amino acid sequence of the wild-type mRNA. This modification is based on the fact that the sequence of the region of the mRNA that is to be translated is important for the efficient translation of a mRNA. The composition and sequence of the various nucleotides is of significance here. In particular, sequences having a high G (guanosine)/C (cytosine) content are more stable than sequences having a high A (adenosine)/U (uracil) content. It is therefore expedient, while retaining the translated amino acid sequence, to vary the codons as compared with the wild-type mRNA in such a manner that they contain more G/C nucleotides. Owing to the fact that several codons code for the same amino acid (so-called “degeneracy of the genetic code”), it is possible to determine the codons that are advantageous for the stability, preferably with maximum G/C content. As a result, a RNA in the injection buffer preferably has a G/C content that is increased by preferably at least 30%, more preferably by at least 50%, yet more preferably by at least 70%, more preferably by 80%, based on the maximum G/C content (that is to say the G/C content after modification of all potential triplets in the coding region without changing the coded amino acid sequence using the degeneracy of the genetic code, starting from the natural sequence, with the aim of maximising the G/C content) and most preferably the maximum G/C content, the maximum G/C content being given by the sequence whose G/C content is maximised without the coded amino acid sequence being changed thereby.

Depending on the amino acid to be coded for by the modified mRNA, there are various possibilities for modifying the mRNA sequence as compared with the wild-type sequence. In the case of amino acids coded for by codons that contain only G or C nucleotides, no modification of the codon is necessary. Examples thereof are codons for Pro (CCC or CCG), Arg (CGC or CGG), Ala (GCC or GCG) and Gly (GGC or GGG).

On the other hand, codons that contain A and/or U nucleotides can be changed by substitution for different codons which code for the same amino acids but do not contain A and/or U. Examples thereof are:
codons for Pro can be changed from CCU or CCA to CCC or CCG;
codons for Arg can be changed from CGU or CGA or AGA or AGG to CGC or CGG;
codons for Ala can be changed from GCU or GCA to GCC or GCG;
codons for Gly can be changed from GGU or GGA to GGC or GGG.

In some cases, although it is not possible to eliminate A and U nucleotides from the codons, it is possible to reduce the A and U content by using codons which have a smaller content of A and/or U nucleotides. Examples thereof are:
the codons for Phe can be changed from UUU to UUC;
codons for Leu can be changed from UUA, UUG, CUU or CUA to CUC or CUG;
codons for Ser can be changed from UCU or UCA or AGU to UCC, UCG or AGC;
the codon for Tyr can be changed from UAU to UAC;
the codon for Cys can be changed from UGU to UGC;
the His codon can be changed from CAU to CAC;
the codon for Gln can be changed from CAA to CAG;
codons for Ile can be changed from AUU or AUA to AUC;
codons for Thr can be changed from ACU or ACA to ACC or ACG;
the codon for Asn can be changed from AAU to AAC;
the codon for Lys can be changed from AAA to AAG;
codons for Val can be changed from GUU or GUA to GUC or GUG;
the codon for Asp can be changed from GAU to GAC;
the codon for Glu can be changed from GAA to GAG,
the stop codon UAA can be changed to UAG or UGA.

The substitutions listed above can be used individually or in all possible combinations for increasing the G/C content of the modified mRNA as compared with the wild-type mRNA (the original sequence). Combinations of the above substitution possibilities, for example, are preferably used:

substitution of all codons coding for Thr in the original sequence (wild-type mRNA) with ACC (or ACG) and substitution of all codons originally coding for Ser with UCC (or UCG or AGC);
substitution of all codons coding for Ile in the original sequence with AUC and substitution of all codons originally coding for Lys with AAG and substitution of all codons originally coding for Tyr with UAC;
substitution of all codons coding for Val in the original sequence with GUC (or GUG) and substitution of all codons originally coding for Glu with GAG and substitution of all codons originally coding for Ala with GCC (or GCG) and substitution of all codons originally coding for Arg with CGC (or CGG);
substitution of all codons coding for Val in the original sequence with GUC (or GUG) and substitution of all codons originally coding for Glu with GAG and substitution of all codons originally coding for Ala with GCC (or GCG) and substitution of all codons originally coding for Gly with GGC (or GGG) and substitution of all codons originally coding for Asn with AAC;
substitution of all codons coding for Val in the original sequence with GUC (or GUG) and substitution of all codons originally coding for Phe with UUC and substitution of all codons originally coding for Cys with UGC and substitution of all codons originally coding for Leu with CUG (or CUC) and substitution of all codons originally coding for Gln with CAG and substitution of all codons originally coding for Pro with CCC (or CCG);

etc.

In the case of a change in the G/C content of the region of the modified mRNA coding for the protein, this will be increased by at least 7% points, more preferably by at least 15% points, likewise more preferably by at least 20% points, yet more preferably by at least 30% points, as compared with the G/C content of the coded region of the wild-type mRNA coding for the protein. It is particularly preferred in this connection to increase the G/C content of the modified mRNA, in particular in the region coding for the protein, to the maximum extent as compared with the wild-type sequence.

It is further preferred to increase the A/U content in the region of the ribosome binding site of the modified mRNA as compared with the A/U content in the region of the ribosome binding site of the corresponding wild-type mRNA. This modification increases the efficiency of the ribosome binding to the mRNA. Effective binding of the ribosomes to the ribosome binding site (Kozak sequence: GCCGCCACCAUGG (SEQ ID NO: 1), the AUG forms the start codon) in turn effects efficient translation of the mRNA. The increase consists in introducing at least one additional A/U unit, typically at least 3, in the region of the binding site, that is to say −20 to +20 from the A of the AUG start codon.

A modification that is likewise preferred relates to a mRNA in which the coding region and/or the 5′- and/or 3′-untranslated region of the modified mRNA has been so changed as compared with the wild-type mRNA that it does not contain any destabilising sequence elements, the coded amino acid sequence of the modified mRNA preferably being unchanged as compared with the wild-type mRNA. It is known that destabilising sequence elements (DSEs) occur, for example, in the sequences of eukaryotic mRNAs, to which destabilising sequence elements signal proteins bind and regulate the enzymatic degradation of the mRNA in vivo. Therefore, for the further stabilisation of the modified mRNA according to the invention, one or more such changes as compared with the corresponding region of the wild-type mRNA can optionally be carried out in the region coding for the protein, so that no or substantially no destabilising sequence elements are present therein. By such changes it is likewise possible according to the invention to eliminate from the mRNA DSEs present in the untranslated regions (3′- and/or 5′-UTR).

Such destabilising sequences are, for example, AU-rich sequences (“AURES”), which occur in the 3′-UTR sections of numerous unstable mRNAs (Caput et al., Proc. Natl. Acad. Sci. USA 1986, 83: 1670 to 1674) as well as sequence motifs which are recognised by endonucleases (e.g. Binder et al., EMBO J. 1994, 13: 1969 to 1980).

Also preferred is a modified mRNA that has a 5′-cap structure for stabilisation. Examples of cap structures which can be used according to the invention are m7G(5′)ppp (5′(A,G(5′)ppp(5′)A and G(5′)ppp(5′)G.

It is also preferable for the modified mRNA to have a poly(A) tail, preferably of at least 25 nucleotides, more preferably of at least 50 nucleotides, yet more preferably of at least 70 nucleotides, likewise more preferably of at least 100 nucleotides, most preferably of at least 200 nucleotides.

Also preferably, the modified mRNA has at least one IRES and/or at least one 5′- and/or 3′-stabilising sequence. According to the invention, one or more so-called IRESs (“internal ribosome entry side”) can be introduced into the modified mRNA. An IRES can thus function as the sole ribosome binding site, but it can also serve to provide a mRNA that codes for a plurality of proteins, peptides or polypeptides which are to be translated, independently of one another, by the ribosomes (“multicistronic mRNA”). Examples of IRES sequences which can be used according to the invention are those from picorna viruses (e.g. FMDV), plague viruses (CFFV), polio viruses (PV), encephalo-myocarditis viruses (ECMV), foot-and-mouth viruses (FMDV), hepatitis C viruses (HCV), classic swine fever viruses (CSFV), murine leukoma virus (MLV), simean immunodeficiency viruses (SIV) or cricket paralysis viruses (CrPV).

It is also preferable for a modified mRNA to have at least one 5′- and/or 3′-stabilising sequence. These stabilising sequences in the 5′- and/or 3′-untranslated regions effect an increase in the half-life of the mRNA in the cytosol. Such stabilising sequences can have 100% sequence homology with naturally occurring sequences, which occur in viruses, bacteria and eukaryotes, but can also be partially or wholly of synthetic nature. As an example of stabilising sequences which can be used in the present invention there may be mentioned the untranslated sequences (UTR) of the globin gene, for example of Homo sapiens or Xenopus laevis. Another example of a stabilising sequence has the general formula (C/U)CCANₓCCC(U/A)PyₓUC(C/U)CC (SEQ ID NO: 2), which is contained in the 3′-UTR of the very stable mRNA that codes for α-globin, (I)-collagen, 15-lipoxygenase or for tyrosine-hydroxylase (see Holcik et al., Proc. Natl. Acad. Sci. USA 1997, 94: 2410 to 2414). Such stabilising sequences can, of course, be used individually or in combination with one naother and also in combination with other stabilising sequences known to a person skilled in the art.

In a preferred embodiment of the present invention, the modified mRNA contains at least one analogue of naturally occurring nucleotides. This/these analogue/analogues serves/serve to further stabilise the modified mRNA, this being based on the fact that the RNA-degrading enzymes occurring in the cells preferentially recognise naturally occurring nucleotides as substrate. By introducing nucleotide analogues into the RNA, therefore, RNA degradation is made more difficult, the introduction of these analogues, in particular into the coding region of the mRNA, having a positive or negative effect on the translation efficiency. There may be mentioned as examples of nucleotide analogues which can be used according to the invention, without implying any limitation, phosphoramidates, phosphorothioates, peptide nucleotides, methyl phosphonates, 7-deazaguanosine, 5-methylcytosine and inosine. The preparation of such analogues is known to a person skilled in the art, for example from U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642. Such analogues can occur in both untranslated and translated regions of the modified mRNA.

In a further preferred embodiment of the present invention, the modified mRNA additionally contains a sequence coding for a signal peptide. This sequence coding for a signal peptide is preferably from 30 to 300 bases long, coding for from 10 to 100 amino acids. More preferably, the sequence coding for a signal peptide is from 45 to 180 bases long, which code for from 15 to 60 amino acids. By way of example, the following sequences mentioned in Table 1 can be used for modifying the RNA used according to the invention. Also included are those sequences mentioned in Table 1 that have from 1 to 20, preferably from 1 to 10 and most preferably from 1 to 5 base substitutions to A, T, C or G in comparison with one of the sequences mentioned in Table 1.

TABLE 1
Examples of signal peptide sequences: amino acid sequences coded for by the first
exon of MHC class I or MHC class II genes, and myelin oligodendrocyte glycoprotein.

| Name of the signal | |
|---|---|
| sequence | Sequence (peptide and nucleotide sequence) |
| HLA-B*07022 | MLVMAPRTVLLLLSAALALTETWAG (SEQ ID NO: 4) |
| | RNA sequence (GC enriched) |
| | AUG CUG GUG AUG GCC CCG CGG ACC GUC CUC CUG CUG CUG |
| | AGC GCG GCC CUG GCC CUG ACG GAG ACC UGG GCC GGC (SEQ ID |
| | NO: 3) |
| HLA-A*3202 | MAVMAPRTLLLLLLGALALTQTWAG (SEQ ID NO: 6) |
| | RNA sequence (GC enriched) |
| | AUG GCC GUG AUG GCG CCG CGG ACC CUG CUC CUG CUG CUG |
| | CUG GGC GCC CUG GCC CUC ACG CAG ACC UGG GCC GGG (SEQ ID |
| | NO: 5) |
| HLA-A*01011 | MAVMAPRTLLLLLSGALALTQTWAG (SEQ ID NO: 8) |
| | AUG GCC GUG AUG GCG CCG CGG ACC CUG CUC CUG CUG CUG |
| | AGC GGC GCC CUG GCC CUG ACG CAG ACC UGG GCC GGG (SEQ ID |
| | NO: 7) |
| HLA-A*0102 | MAVMAPRTLLLLLSGALALTQTWAG (SEQ ID NO: 10) |
| | AUG GCC GUG AUG GCG CCG CGG ACC CUG CUC CUG CUG CUG |
| | AGC GGC GCC CUG GCC CUG ACG CAG ACC UGG GCC GGG (SEQ ID |
| | NO: 9) |
| HLA-A*0201 | MAVMAPRTLVLLLSGALALTQTWAG (SEQ ID NO: 12) |
| | AUG GCC GUG AUG GCG CCG CGG ACC CUG GUC CUC CUG CUG |
| | AGC GGC GCC CUG GCC CUG ACG CAG ACC UGG GCC GGG (SEQ ID |
| | NO: 11) |
| HLA-A*0301 | MAVMAPRTLLLLLSGALALTQTWAG (SEQ ID NO: 14) |
| | AUG GCC GUG AUG GCG CCG CGG ACC CUG CUC CUG CUG CUG |
| | AGC GGC GCC CUG GCC CUG ACG CAG ACC UGG GCC GGG (SEQ ID |
| | NO: 13) |
| HLA-A*1101 | MAVMAPRTLLLLLSGALALTQTWAG (SEQ ID NO: 16) |
| | AUG GCC GUG AUG GCG CCG CGG ACC CUG CUC CUG CUG CUG |
| | AGC GGC GCC CUG GCC CUG ACG CAG ACC UGG GCC GGG (SEQ ID |
| | NO: 15) |
| HLA-B*070201 | MLVMAPRTVLLLLSAALALTETWAG (SEQ ID NO: 18) |
| | AUG CUG GUG AUG GCC CCG CGG ACC GUC CUC CUG CUG CUG |
| | AGC GCG GCC CUG GCC CUG ACG GAG ACC UGG GCC GGC (SEQ ID |
| | NO: 17) |
| HLA-B*2702 | MRVTAPRTLLLLLWGAVALTETWAG (SEQ ID NO: 20) |
| | AUG CGG GUG ACC GCC CCG CGC ACG CUG CUC CUG CUG CUG |
| | UGG GGC GCG GUC GCC CUG ACC GAG ACC UGG GCC GGG (SEQ ID |
| | NO: 19) |
| HLA-Cw*010201 | MRVMAPRTLILLLSGALALTETWACS (SEQ ID NO: 22) |
| | AUG CGG GUG AUG GCC CCG CGC ACC CUG AUC CUC CUG CUG |
| | AGC GGC GCG CUG GCC CUG ACG GAG ACC UGG GCC UGC UCG |
| | (SEQ ID NO: 21) |
| HLA-Cw*02021 | MRVMAPRTLLLLLSGALALTETWACS (SEQ ID NO: 24) |
| | AUG CGG GUG AUG GCC CCG CGC ACC CUG CUC CUG CUG CUG |
| | AGC GGC GCG CUG GCC CUG ACG GAG ACC UGG GCC UGC UCG |
| | (SEQ ID NO: 23) |
| HLA-E*0101 | MVDGTLLLLSSEALALTQTWAGSHS (SEQ ID NO: 26) |
| | AUG GUG GAC GGC ACC CUG CUC CUG CUG AGC UCG GAG GCC |
| | CUG GCG CUG ACG CAG ACC UGG GCC GGG AGC CAC AGC (SEQ ID |
| | NO: 25) |
| HLA-DRB1 | MVCLKLPGGSCMTALTVTLMVLSSPLALA (SEQ ID NO: 28) |
| | AUG GUG UGC CUG AAG CUC CCG GGC GGG AGC UGC AUG ACC |
| | GCC CUG ACG GUC ACC CUG AUG GUG CUG UCG AGC CCC CUG |
| | GCG CUG GCC (SEQ ID NO: 27) |
| HLA-DRA1 | MAISGVPVLGFFIIAVLMSAQESWA (SEQ ID NO: 30) |
| | AUG GCC AUC AGC GGC GUG CCG GUC CUG GGG UUC UUC AUC |
| | AUC GCG GUG CUC AUG UCG GCC CAG GAG AGC UGG GCC (SEQ ID |
| | NO: 29) |
| HLA-DR4 | MVCLKFPGGSCMAALTVTLMVLSSPLALA (SEQ ID NO: 32) |
| | AUG GUG UGC CUG AAG UUC CCG GGC GGG AGC UGC AUG GCC |
| | GCG CUC ACC GUC ACG CUG AUG GUG CUG UCG AGC CCC CUG |
| | GCC CUG GCC (SEQ ID NO: 31) |
| Myelin oligodendrocyte | MACLWSFSWPSCFLSLLLLLLLQLSCSYA (SEQ ID NO: 34) |
| glycoprotein | AUG GCC UGC CUG UGG AGC UUC UCG UGG CCG AGC UGC UUC |
| | CUC AGC CUG CUG CUG CUG CUG CUC CUG CAG CUG AGC UGC |
| | AGC UAC GCG (SEQ ID NO: 33) |

Various processes are known to a person skilled in the art for carrying out the above-described modifications. For example, for the substitution of codons in the modified mRNA according to the invention it is possible in the case of relatively short coding regions to synthesise the entire mRNA chemically using standard techniques. Substitutions, additions or eliminations of bases are preferably introduced, however, using a DNA matrix for the preparation of the modified mRNA with the aid of techniques of conventional target-oriented mutagenesis (see e.g. Maniatis et al., Molecular Cloning: A Laboratory Manual, Cold Spring Harbor Laboratory Press, 3rd ed., Cold Spring Harbor, N.Y., 2001). In such a process, a corresponding DNA molecule is transcribed in vitro in order to prepare the mRNA. This DNA matrix has a suitable promoter, for example a T7 or SP6 promoter, for the in vitro transcription, followed by the desired nucleotide sequence for the mRNA that is to be prepared and a termination signal for the in vitro transcription. The DNA molecule forming the matrix of the RNA construct to be prepared can be prepared by fermentative propagation and subsequent isolation as part of a plasmid replicatable in bacteria. Thus, the desired nucleotide sequence can be cloned into a suitable plasmid according to methods of molecular biology known to a person skilled in the art, using short synthetic DNA oligonucleotides which have short single-stranded transitions at the resulting cleavage sites, or using genes prepared by chemical synthesis (see Maniatis et al., supra). The DNA molecule is then cut out of the plasmid, in which it can be present in a single copy or in multiple copies, by digestion with restriction endonucleases.

The above-described modifications of the RNA, in particular mRNA, can occur within the scope of the invention individually or in combination with one another. Likewise, one or more modification(s) can be combined with the above-described complexing of the RNA, in particular mRNA.

The aim of the invention is to increase RNA transfer and/or RNA translation in a host organism. A host organism within the scope of the invention is to be understood as being any organism into whose cells or tissue RNA can be transferred, followed by the translation thereof. A host organism within the scope of the invention is in particular a mammal selected from the group consisting of mouse, rat, pig, cow, horse, dog, cat, ape and, in particular, human.

With the present invention it is shown that luciferase-coding RNA, in particular mRNA, diluted in the RL injection buffer according to the invention (with or without lactate) gives a significantly higher translation rate than mRNA that has been diluted in standard buffers conventionally used for RNA, such as HBS or PBS (see FIG. 1). Furthermore, it is shown that the efficiency of transfer and translation of injected mRNA is dependent to a large degree on the presence of calcium ions. In corresponding comparative tests with/without calcium ions in the RL injection buffer (with or without lactate), it was found that the absence of calcium significantly reduces the efficiency of the RNA transfer to a level that is comparable with that of the standard buffers PBS and HBS (see FIG. 2).

It has therefore been found that, firstly, a RL injection buffer according to the invention (with or without lactate) considerably increases RNA transfer and, secondly, that this improved RNA transfer is increased by yet a further factor by a RL injection buffer according to the invention (with or without lactate) having a high calcium concentration of up to 100 mM.

The injection buffer according to the invention is preferably used in combination with RNA in a RNA injection solution. The invention therefore further provides a RNA injection solution containing RNA and an injection buffer which contains at least 50 mM sodium chloride (NaCl), at least 0.01 mM calcium chloride (CaCl₂)) and optionally at least 3 mM potassium chloride (KCl), for increasing RNA transfer and/or RNA translation in cells. Preference is given to a RNA injection solution according to the invention in which the injection buffer contains at least from 50 mM to 800 mM, preferably at least from 60 mM to 500 mM, more preferably at least from 70 mM to 250 mM, particularly preferably from 60 mM to 110 mM, sodium chloride (NaCl), at least from 0.01 mM to 100 mM, preferably at least from 0.5 mM to 80 mM, more preferably at least from 1.5 mM to 40 mM, calcium chloride (CaCl₂)) and optionally at least from 3 mM to 500 mM, preferably at least from 4 mM to 300 mM, more preferably at least from 5 mM to 200 mM, potassium chloride (KCl).

The injection buffer of the RNA injection solution according to the invention preferably further contains lactate. Such an injection buffer of the RNA injection solution according to the invention preferably contains at least 15 mM lactate. Preference is given further to a RNA injection solution according to the invention in which the injection buffer contains from 15 mM to 500 mM, preferably from 15 mM to 200 mM, more preferably from 15 mM to 100 mM, lactate.

The RNA injection solution can be prepared according to any desired process from the prior art. Preferably, the RNA is diluted in the RL injection buffer or RL injection buffer with lactate. Likewise, the RNA can be used in the form of dry (for example freeze-dried) RNA, and the RNA injection buffer or RL injection buffer with lactate can be added thereto, optionally with an increase in temperature, stirring, ultrasound, etc., in order to accelerate dissolution. The concentration ratios are to be chosen in accordance with the particular conditions (host organism, in particular mammal, into which the RNA injection solution is injected, state of health, age, etc. of the host organism, etc.).

The RNA in the RNA injection solution according to the invention is preferably naked RNA, more preferably mRNA, preferably naked mRNA, as already defined hereinbefore.

As described, the RNA injection solution according to the invention can be used in particular for increasing RNA transfer and RNA translation into/in a host organism.

Accordingly, the present invention further provides the use of the above-described RNA injection solution for increasing RNA transfer and/or RNA translation into/in a host organism.

The dosage (in respect of amount and duration for clinical applications in particular) of the RNA to be transferred in RL injection buffer (with or without lactate) has also been investigated. The investigations revealed an increase in luciferase expression as the amounts of mRNA increased up to 0.1 μg (in 100 μl injection volume) in mice and up to 3 mg (in 150 μl injection volume) in humans. The translation of mRNA takes place transiently and is consequently regulated so that, for a lasting, uniform expression of the foreign molecule (protein), a repeat injection, dependent on various factors, such as the foreign molecule to be expressed and the intended action, the organism receiving the injection, as well as the state (of health) thereof, etc., should be carried out approximately every three days, but even every two days or daily. The amount of RNA—likewise in dependence on various factors, inter alia those mentioned above—can be from 0.01 μg to 1000 μg, preferably from 1 μg to 800 μg, likewise preferably from 2 μg to 500 μg, more preferably from 5 μg to 100 μg, yet more preferably from 10 μg to 90 μg, most preferably from 20 μg to 80 μg, in 100 μl injection volume. The amount of RNA is particularly preferably 60 μg in 100 μl injection volume.

Uses according to the invention both of the RNA and of the RL injection buffer or RL injection buffer with lactate, and of the RNA injection solution of the present invention, are accordingly, for example, use in the treatment and/or prophylaxis of, or in the preparation of a medicament for the treatment and/or prophylaxis of, cancer or tumour diseases, for example melanoma, such as malignant melanoma, skin melanoma, carcinoma, such as colon carcinoma, lung carcinoma, such as small-cell lung carcinoma, adenocarcinoma, prostate carcinoma, oesophageal carcinoma, breast carcinoma, renal carcinoma, sarcoma, myeloma, leukaemia, in particular AML (acute myeloid leukaemia), glioma, lymphomas and blastomas, allergies, autoimmune diseases, such as multiple sclerosis, viral and/or bacterial infections.

For example, the present invention includes the use both of the RNA and of the RL injection buffer or RL injection buffer with lactate, and also of the RNA injection solution, inter alia for gene therapy and for vaccination, for example for anti-viral or tumour vaccination, for the prevention of the diseases mentioned above.

A “gene therapy” within the scope of the present invention means especially the restoration of a missing function of the body or of the cell by the introduction of a functioning gene into the diseased cells, or the inhibition of an impaired function by corresponding genetic information. For example, in the case of a tumour suppressor gene, for example p53, that is missing or that is expressed in only small amounts, this can be introduced into the cell in the form of its mRNA and inserted into the DNA, and the originally deficiently expressed protein can thus be produced in physiologically relevant amounts again. Examples of tumour suppressor genes within the scope of the present invention are p53 TP53, RB1, APC, WT1, NF1, NF2, VHL, BRCA1, BRCA2, DCC, MEN 1, MEN 2, PTCH, p57/KIP2, MSH2, MLH1, FMS1, FMS2, MET, p16/INK4a/CDKN2, CDK4, RET, EXT1, EXT2, EXT3, PTEN/MMAC1, ATM, BLM, XPB, XPD, XPA, XPG, FACC, FACA, SMAD4/DPC4, p14^{Art}(p19^{Art}), DPC4, E-CAD, LKB1/STK1, TSC2, PMS1, PMS2, MSH6, TGF-β type II R, BAX, α-CAT, MADR2/SMAD2, CDX2, MKK4, PP2R1B, MCC, etc.

A vaccination within the scope of the invention means the introduction of genetic information in the form of RNA, in particular mRNA, into an organism, in particular into one/several cell/cells or tissue of the organism. The mRNA so administered is translated in the organism the target molecule (e.g. peptide, polypeptide, protein), that is to say the target molecule coded for by the mRNA is expressed and triggers an immune response. It is known that antigen-presenting cells (APCs) play an obligatory key role during the triggering of an immune response, because they are the only cell type in which, on activation, all signals necessary for the initiation of the proliferation of antigen-specific immune cells are triggered. A vaccination within the scope of the present invention can be carried out, for example, by using RNA, in particular mRNA, which codes for an antigen, the antigen being a tumour antigen in the case of a tumour vaccination or a foreign antigen in the case of a vaccine against foreign pathogens. Examples of tumour antigens according to the present invention are T-cell-defined tumour antigens, such as, for example, “cancer/testis” antigens, e.g. MAGE, RAGE, NY-ESO-1, differentiation antigens, e.g. MART-1/Melan-A, tyrosinase, gp100, PSA, CD20, antigenic epitopes of mutated genes, e.g.: CDK4, caspase-8 or oncofetal antigens, e.g. CEA, AF. Other tumour antigens are, for example, tumour antigens CD5 and CAMPATH-1(CDw52), which occur in T-cell and B-cell lymphomas, CD20, which occur in non-Hodgkin's B-cell lymphomas, the tumour antigens CEA (carcinoembryogenic antigen), mucin, CA-125 and FAP-a, which occur in solid tumours, in particular in epithelial tumours (breast, intestine and lung), tenascin, and metalloproteinases, which additionally occur in glioblastoma tumours. Further tumour antigens are, for example, the tumour antigens EGF (epidermal growth factor), p185HER2 and the IL-2 receptor, which occur in lung, breast, head and neck as well as T- and B-cell tumours, or the tumour antigen SV40, etc.

It is also possible to a RNA, in particular mRNA, that codes for a plurality of such antigens. As a result, a melanoma, carcinoma, AML or glioma can effectively be controlled, because a combination of different antigens specific for the particular tumour has an extremely broad spectrum of action. The RNA, in particular mRNA, of the invention can further code for an immunogenic protein. Such an immunogenic protein can mediate the reactivation of an immune response. Such a reactivation is based on the finding that almost every organism has so-called “memory immune responses” to certain foreign molecules, e.g. proteins, in particular viral proteins, antigens. This means that an organism has already been infected with such a foreign molecule at an earlier time and that an immune response to that foreign molecule, for example a viral protein, has already been triggered by this infection, and this response remains in the “memory”, that is to say it is stored. When the organism is infected with the same foreign molecule again, the immune response is reactivated. According to the invention, such a reactivation of the immune response can be effected by vaccination with a RNA, in particular mRNA, which contains at least one region coding for at least one immunogenic protein. Preference is given to a RNA, in particular mRNA, that codes both for one or more antigens and for one or more immunogenic proteins.

Immunogenic proteins within the scope of the invention are preferably structural proteins of viruses, in particular matrix proteins, capsid proteins and surface proteins of the lipid membrane. Further examples of such viral proteins are proteins of adenoviruses, rhinoviruses, corona viruses, retroviruses. Particular preference is given here to the hepatitis B surface antigen (referred to as “HBS antigen” hereinbelow) and influenza matrix proteins, in particular the influenza matrix M1 protein.

The present invention relates further to the use of RNA and of the above-described RL injection buffer or RL injection buffer with lactate, or of the above-described RNA injection solution, for increasing the RNA transfer and/or RNA translation of RNA in “in vitro” processes, for example for gene expression analyses or for in vitro screening processes, e.g. by HTS (high throughput screening).

The present invention further provides a method for increasing the RNA transfer and/or RNA translation of RNA in a host organism, for example for the treatment and/or prophylaxis of cancer or tumour diseases, for example melanoma, such as malignant melanoma, skin melanoma, carcinoma, such as colon carcinoma, lung carcinoma, such as small-cell lung carcinoma, adenocarcinoma, prostate carcinoma, oesophageal carcinoma, breast carcinoma, renal carcinoma, sarcoma, myeloma, leukaemia, in particular AML (acute myeloid leukaemia), glioma, lymphomas and blastomas, allergies, autoimmune diseases, such as multiple sclerosis, viral and/or bacterial infections, and for gene therapy and/or vaccination, optionally for anti-viral vaccination, for the prevention of the above-mentioned diseases, the method comprising the following steps:
a.) preparation of a RNA injection solution of the present invention and
b.) administration of the RNA injection solution from step a.) to a host organism.

The preparation of the RNA injection solution from step a. can be carried out as described above, that is to say according to any desired process from the prior art, preferably by diluting the RNA in the RL injection buffer or RL injection buffer with lactate. Here too, the concentration ratios are to be chosen in dependence on the above-described conditions (e.g. host organism, in particular mammal, into which the RNA injection solution is injected, state of health, age, etc. of the host organism, etc.). The RNA injection solution can be administered, for example, by means of an injection syringe (e.g. Sub-Q, Becton Dickinson, Heidelberg, Germany) in any suitable manner, for example intradermally, intraepithelially, subcutaneously, intravenously, intravasally, intraarterially, intraabdominally, intraperitoneally, intranodally (e.g. into the lymph nodes), etc.

A host organism of the method according to the invention is preferably a mammal selected from the group consisting of mouse, rat, pig, cow, horse, dog, cat, ape and, in particular, human.

The injection solution prepared according to the present invention can, however, also be used for the in vitro transfection of cells with RNA, in particular mRNA. This in vitro transfection can be suitable for laboratory use or can be part of an ex vivo gene therapy, that is to say the removal of cells from a patient, the ex vivo transfection of RNA contained in an injection solution according to the invention, and then retransplantation into the patient. The transfection can be carried out with the aid of an electroporation process, optionally also with the application of voltage pulses with a field strength of not more than from 2 to 10 kVcm⁻¹ and of pulse durations of from 10 to 200 μs and a current density of at least 2 Acm⁻². Provided it is not required for the transfection, longer pulse times in the range from 1 to 100 ms can also be used, however. If the injection solution according to the invention is used for laboratory purposes, all conceivable laboratory cell lines can be transfected with RNA in this manner. For ex vivo gene therapy, numerous cell types come into consideration for transfection, in particular primary human blood cells, pluripotent precursor blood cells, as well as fibroblasts, neurons, endothelial cells or muscle cells, this list being given by way of example and not being intended to be limiting.

All the literature references cited in the present application are incorporated into the present application in their entirety.

The figures and examples below serve to explain and illustrate the present invention further, without limiting it thereto.

### FIGURES

In the experiments shown in FIGS. 1 to 5, a volume of 100 μl of the buffer indicated in each case (compositions of the buffers are given hereinbelow under Materials, 1. Injection buffers), containing mRNA (FIG. 4, pDNA in 100 μl of PBS) coding for Photinus pyralis luciferase, was injected intradermally into the ear muscle of BALB/c mice¹³. The luciferase activity of a complete mouse ear was analysed. This is indicated in million(s) luciferase molecules. The detection limit is shown in the diagrams by a thick line in which a number is given. Each point in the diagrams shows the luciferase expression of a single ear. Short bars with figures indicate the mean values of the various groups. p values are given for groups that differ significantly in their mean value (according to the Mann-Whitney test). In experiments of FIGS. 1, 2 and 5, the ears were removed 15 hours after the injection. The data shown result from at least three independent experiments for each group.

FIG. 1 shows a comparison of different injection buffers for mRNA: phosphate-buffered saline (PBS) and HEPES-buffered saline (HBS) and RL injection buffer with lactate (RL). lacZ mRNA is used as negative control. It was shown according to the invention that mRNA diluted in RL injection buffer with lactate gave a significantly higher (p<0.001) expression of luciferase than mRNA diluted in HBS or PBS (FIG. 1A).

FIG. 2 shows the influence of the absence of calcium (—CaCl), potassium (—KCl) or sodium lactate (—NaLa) in the RL injection buffer (with lactate and without lactate as well as with and without calcium or potassium) on the efficiency of the uptake of the mRNA. The main difference between PBS and HBS as compared with RL injection buffer or RL injection buffer with lactate (with and without calcium) is in the absence of lactate and calcium (in HBS or PBS). Therefore, investigations were carried out in which the transfer and translation of mRNA coding for luciferase were compared using on the one hand complete RL injection buffer (RL injection buffer with lactate) and on the other hand formulations of RL injection buffer without calcium or without potassium or without lactate. These investigations showed that the absence of lactate gave luciferase expression which is comparable with the expression using complete RL injection buffer (RL injection buffer with lactate). By contrast, the absence of calcium in the RL injection buffer or RL injection buffer with lactate lowered the efficiency of the RNA transfer significantly (p=0.004) to a level comparable with that of PBS and HBS.

FIGS. 3 and 4 show the kinetics of the mRNA translation directly in vivo. Parallel kinetics experiments with RNA in RL-with lactate according to the invention and with DNA in PBS standard buffer were carried out and compared. The translation of mRNA (in RL injection buffer with lactate) (FIG. 3) or pDNA (in PBS) (FIG. 4) for ten days after the injection was recorded and is shown in the diagrams. In both test procedures (RNA and DNA), the luciferase activity in living mice was recorded. The results of a representative ear are shown. The expression of luciferase detected after the injection of (luciferase-coding) mRNA in RL injection buffer with lactate reached its maximum very early (17 hours) and was no longer detectable after nine days (FIG. 3). By contrast, the injection of (luciferase-coding) pDNA in PBS resulted in a later protein expression, which reached its maximum three days after the injection and lasted for more than nine days (FIG. 4). These results again confirm not only the efficiency of the RL injection buffer with lactate according to the invention but also that RNA is far more suitable as a vehicle for transient gene expression in host organisms, in particular mammals, than DNA. RNA is expressed on the one hand more quickly and on the other hand transiently, meaning that the desired gene expression can be triggered earlier and for a limited time, and accordingly in a more targeted and differentiated manner. With these investigations it was possible to demonstrate both the increased, successful RNA transfer and the effective subsequent translation.

FIG. 5 shows the effect of different amounts of mRNA on luciferase expression. These experiments were carried out in particular in order to determine more precisely the dosage of the RNA to be transferred in the RL injection buffer with lactate, in particular for clinical applications, in respect of amount and duration. To this end, increasing amounts of RNA were injected into a plurality of mice. An increase in luciferase expression was detected with increasing amounts of mRNA up to 5 μg (in 100 μl injection volume). Dosages higher than 5 μg did not lead to a further improvement in the expression. In corresponding experiments in humans (not shown), an amount of 120 μg of mRNA was used, which was increased to 200 μg and resulted in improved expression. The amount of 200 μg of mRNA in humans, compared with 5 μg in the mouse, can be derived inter alia from the size of the injection site, which is approximately 40 times as large in humans. The human experiments were carried out on healthy volunteers, after explaining the background and possible consequences of the investigations and after consent had been given.

With regard to the dosage over time, it should be noted that the translation of mRNA takes place transiently (as shown in FIG. 3, it reaches its maximum after 12 hours and is no longer detectable after nine days) and is consequently regulated. For a lasting, uniform translation of the foreign molecule (protein), therefore, a repeat injection approximately every day, every two days or every three days (depending on factors such as, for example, the foreign molecule or the organism into which the mRNA is injected) is suitable.

FIG. 6 again shows the influence of CaCl₂ on the luciferase activity. To this end, serial dilutions of CaCl₂ in luciferase lysis buffer (the final concentrations are given in the diagram) were prepared and the same defined amount of recombinant luciferase protein was added to all the samples (final concentration about 4.7 pM). The light emission of the mixtures was tested with a luminometer (after addition of ATP and luciferin). The influence of the CaCl₂ concentration on the luciferase activity was then calculated according to the following formula:

% relative luciferase activity(RLA)=(RLA of the sample with defined CaCl₂concentration−RLA of the pure lysis buffer)/(RLA of the sample without CaCl₂−RLA of the pure lysis buffer)×100%.

The presence of Ca²⁺ ions at a relatively high concentration (from about 2 mM) did not increase the enzymatic activity of luciferase.

FIG. 7 again shows the influence of the CaCl₂ concentration on the mRNA transfer in vivo. Various concentrations of RL injection buffer with lactate were used in order to prepare RNA injection solutions (100 μl) having the same amount of mRNA coding for Photinus pyralis luciferase (20 μg) but having different osmolarities (osmol.). The RNA injection solutions were injected into the ear muscles of BALB/c mice. After 15 hours, the mice were sacrificed and lysates of the ears were prepared. The calculated total amount of luciferase molecules produced per ear, the mean value of the various groups (bars with numbers), the size of each group (n) and the detection limit of the test (thick line with a number) are shown. As the result it was found that an efficient transfer, and hence an efficient subsequent translation, of mRNA in vivo requires a minimum ion concentration of 170 mOsm.

FIGS. 8A-E show the characterisation of cells which express the supplied mRNA in vivo. 20 μg of mRNA coding for Escherichia coli β-galactosidase, diluted in a total volume of a RNA injection solution containing 100 μl of RL injection buffer with lactate were injected. 14 hours after the injection, the mice were sacrificed, the ears were removed and transverse cryosections were prepared. The sections shown in FIGS. 8A and 8C to 8E are characterised by colour.

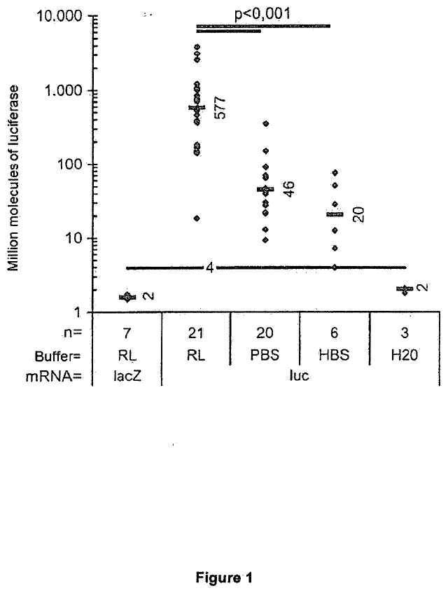

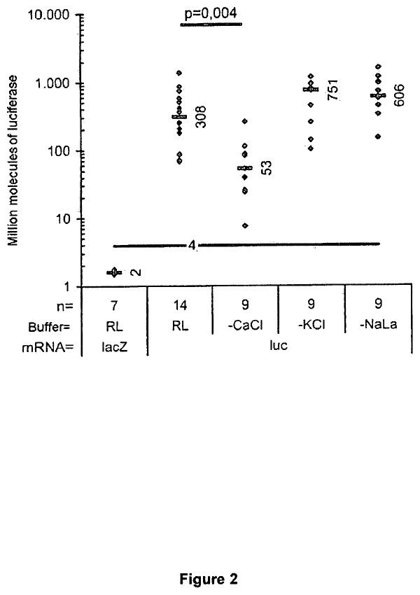

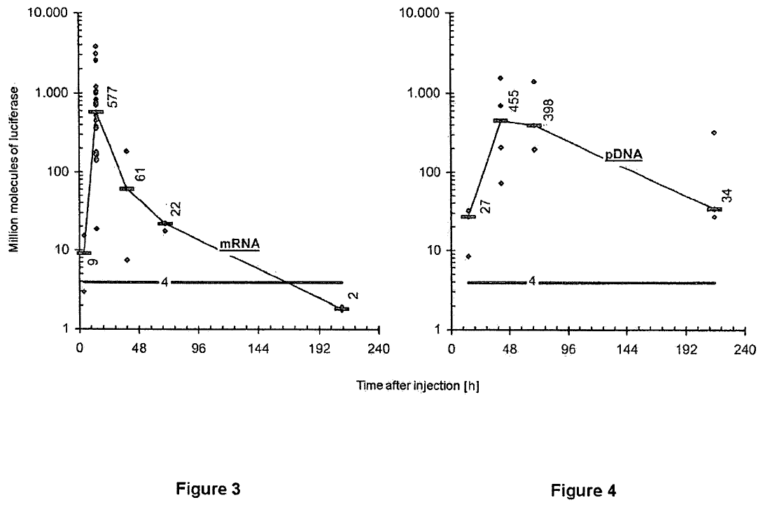

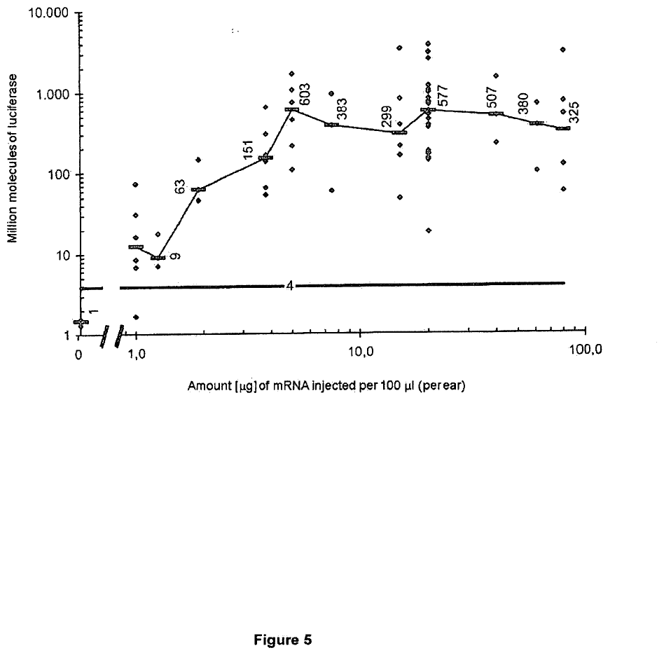

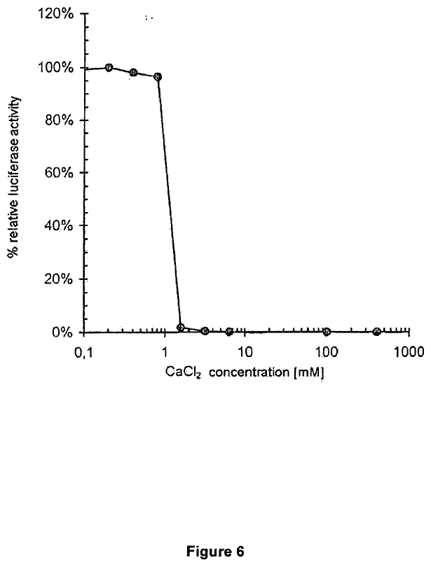

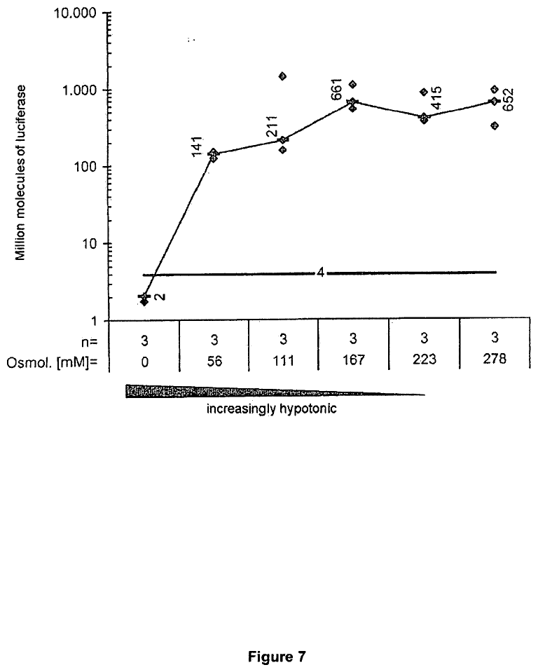

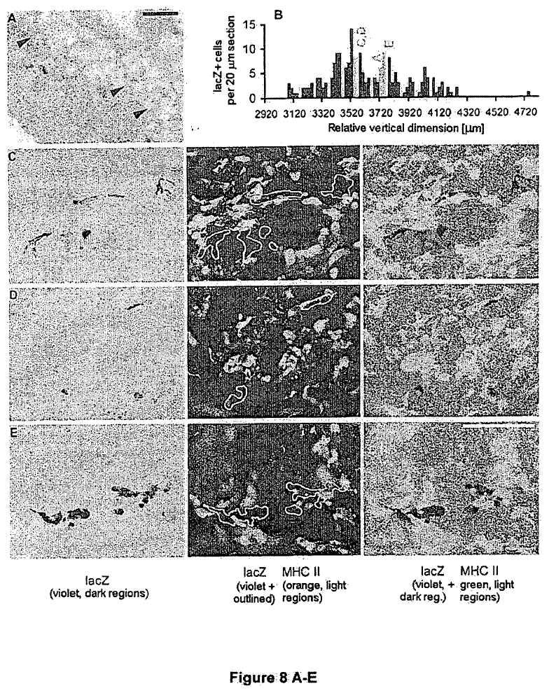

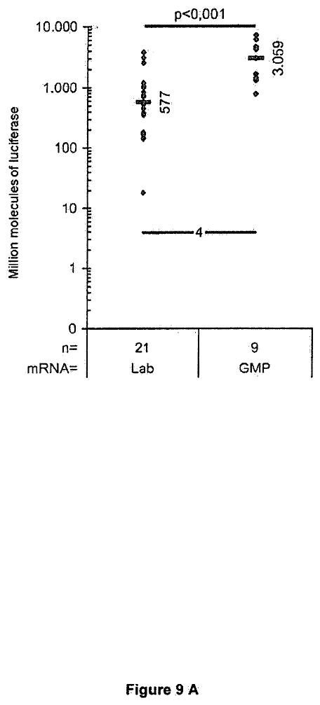

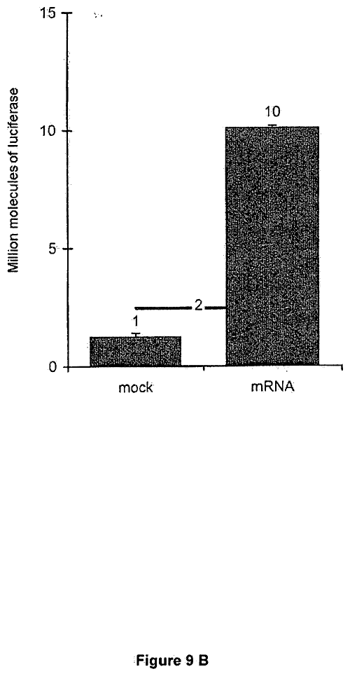

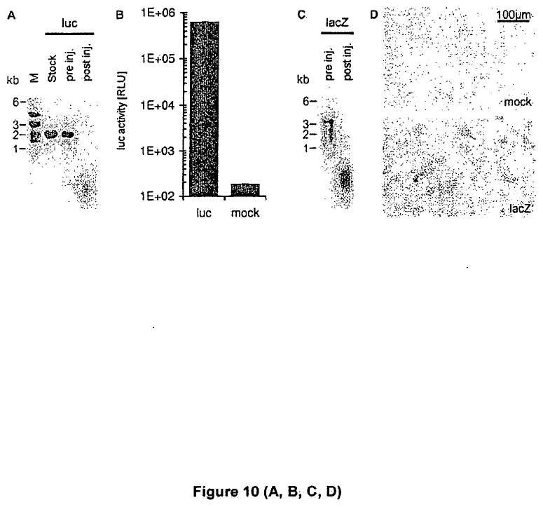

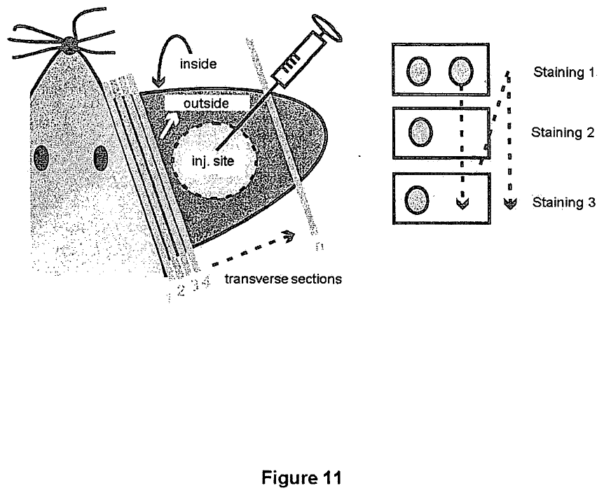

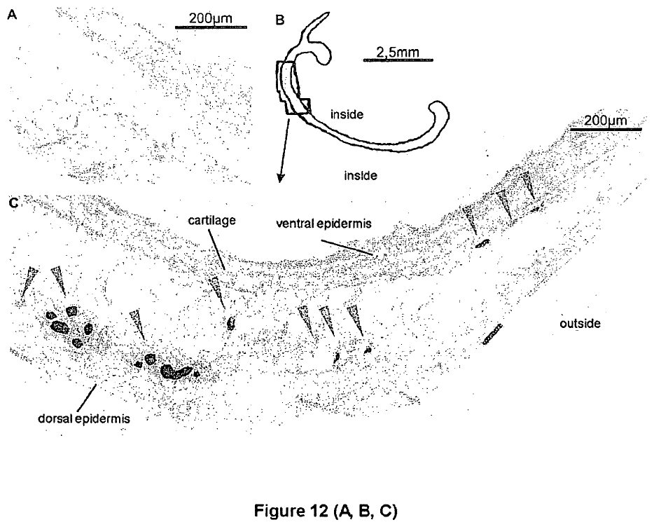

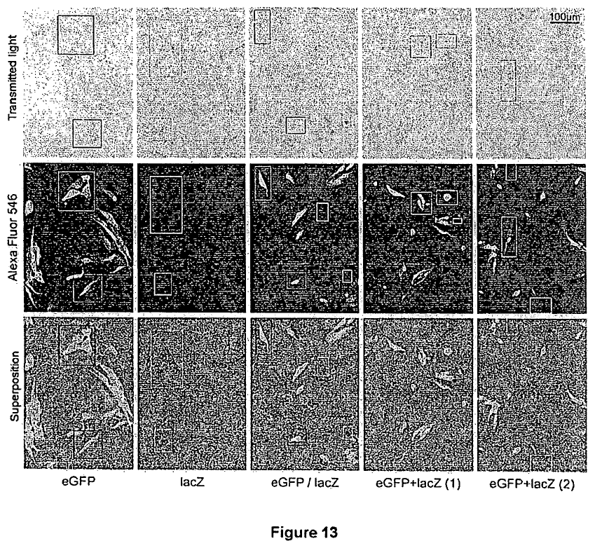

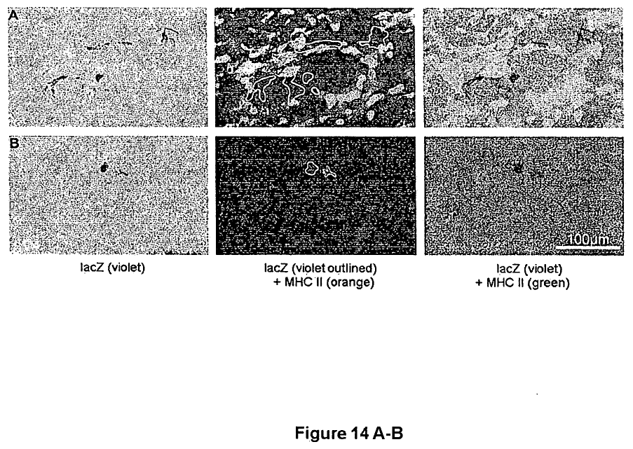

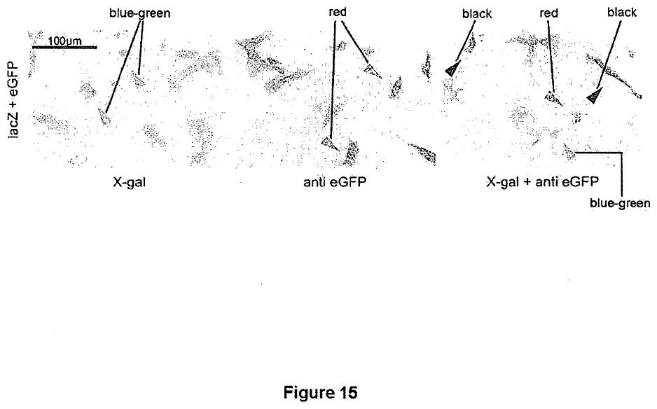

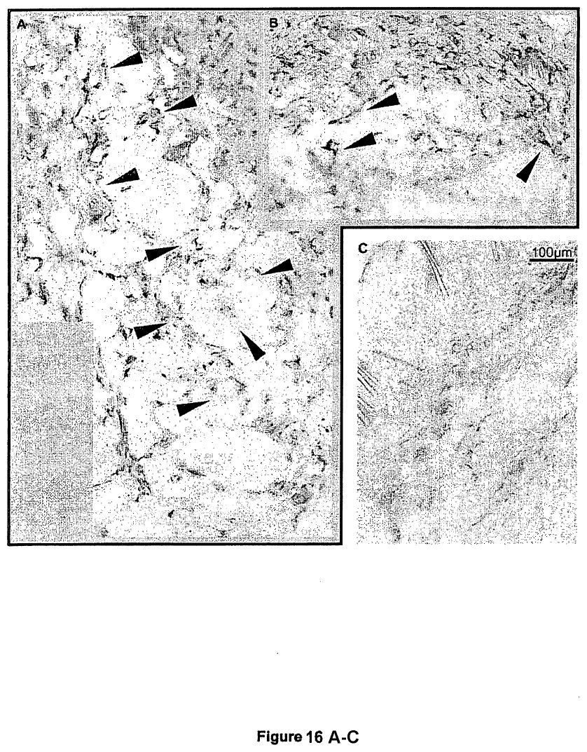

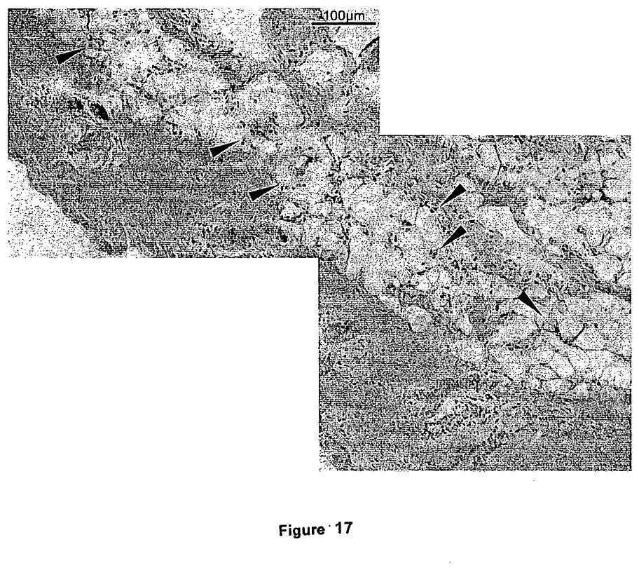

Furthermore, a directed gene expression of RNA in RL injection buffer (with or without lactate) was investigated. To this end, which cell types take up and translate the exogenous RNA transferred in RL injection buffer (with or without lactate) was defined (see also Example 5, FIGS. 8 A-E and, analogously, FIGS. 11, 12, 16 and 17). Thereafter, it was analysed how, within the scope of a mRNA-based vaccination according to the invention, an immune response can be triggered by the translation of exogenous RNA transferred in RL injection buffer (with or without lactate) in defined target cells of the immune system.

In experiments, shown in FIG. 8A, each fifth individual section was stained with X-gal-containing solution. Up to 10 β-galactosidase positive cells were detected in successive 20 μm thick cryosections. The field of expression, that is to say β-galactosidase positive cells (indicated by arrows), covered one to two millimetres in the longitudinal direction and sagittal direction of the ear and was localised in a narrow layer between the epidermis and the cartilage of the ear muscle.

According to the invention it was further investigated whether APCs detect a foreign antigen by direct uptake and self-translation of the transferred mRNA or by the uptake of the translation product of the transferred RNA by other cells (so-called “cross presentation”). Owing to the localisation of the cells, their shape and their MHC class II phenotype, it was possible to conclude that cells that take up and express exogenous naked mRNA at the injection site are principally muscle cells and/or fibroblasts (FIG. 8A). The results correspond with the above-mentioned “cross presentation” of antigens which were translated by other cells. Such a procedure would likewise explain the formation of antibodies against the proteins coded for by nucleic acid vaccines. According to the invention it was thus for the first time possible to ascertain that the triggering of the immune response accordingly takes place via a so-called “cross priming”, in that muscle cells or dermis cells (fibroblasts) take up and express the transfer RNA, and the APCs are activated by these cells.

The histogram in FIG. 8B shows the number of β-galactosidase positive cells in successive sections. Each bar represents one section.

In experiments of FIGS. 8C to 8E, each fifth individual section was stained, namely for MHC class II expression (detected by Alexa 546 immunofluorescence staining, green) and β-galactosidase expression (detected by magenta-gal staining, violet). The images in the left-hand column show a magneta-gal staining—alone—with β-galactosidase positive cells in violet=(deep)dark regions. In the middle and right-hand column, the superposition of a magenta-gal staining (shown by regions of interest in the middle and in the right-hand column) and MHC class II staining—alone—(orange=light regions in the middle column) (green=light regions in the right-hand column). As will be seen, most β-galactosidase positive cells clearly appear to be MHC class II negative.

FIGS. 9A-B show the in vivo transfer of naked mRNA in the mouse and in humans. mRNA coding for luciferase was prepared and dissolved in RL injection solution containing RL injection buffer. The detection limit is shown in the diagrams by a thick line with a number.

In the experiments shown in FIG. 9A, a total amount of 100 μl, containing 20 μg of mRNA, was injected into the ear muscle of mice. 14 hours after the injection, the mice were sacrificed, the cells of the ears were lysed, and the lysate was investigated for luciferase expression. The number of luciferase molecules per ear was calculated (recombinant luciferase was used as standard). The data come from at least three independent experiments for each group.

In the experiments shown in FIG. 9B, the same mRNA (120 μg) in a total volume of 200 μl was injected into human skin (into the leg of volunteers). 16 hours later, biopsies having a diameter of 2 mm were taken (stamped out) under local anaesthetic, namely from the middle of the injection site (“mRNA”) and at a distance from the injection site (“mock”). Luciferase activity could only be detected in the middle of the injection site. One of two independent experiments is shown. With these results, the direct transfer of naked mRNA in vivo into human skin could be demonstrated. Accordingly, the invention permits efficient directed mRNA-based vaccination in humans.

FIGS. 10 A-D show the integrity and translation capacity of the injected mRNA in RL injection buffer with lactate. The integrity was tested using formaldehyde-agarose gel electrophoresis (1.2% w/v). To this end, 1 μg of mRNA coding either for Photinus pyralis luciferase (luc, 1.9 kB, FIG. 10A) or for Escherichia coli β-galactosidase (lacZ, 3.5 kB, FIG. 10C) was separated. No difference in the integrity of the mRNA (before the injection) before dilution in the respective injection buffer (stock solution) and after dilution in the respective injection buffer was detected. By contrast, visible, complete degradation of the mRNA (after the injection) is to be detected when the residues of the RNA injection solution are collected from the injection syringe. These residues have evidently been contaminated with ribonucleases by contact of the injection syringe with the mouse or human tissue.

The translation capacity of the injected mRNA was tested by electroporation of BHK21 cells with 10 μg of mRNA. There were used as control either 10 μg of irrelevant mRNA or no mRNA (mock). The cells were subsequently either lysed and their luciferase activity investigated with a luminometer (FIG. 10B) or were stained with X-gal and their luciferase activity investigated with a light microscope (FIG. 10D).

FIG. 11 shows the identification of the mRNA transfer at cell level. The diagram shows the view of a mouse. In the diagram, the outer (dorsal) side is directly visible to the viewer. mRNA in RL injection buffer with lactate was injected into the ear muscle of the mouse. Successive transverse sections of the ear (1, 2, 3, 4) were prepared. The sections were collected in various sets (1, 2, 3, 4), dried in air and stored at −20° C. until the various staining operations.

FIGS. 12A-C show the transfer of the mRNA at cell level. 5 μg of mRNA coding for Escherichia coli β-galactosidase in RL injection buffer with lactate were injected into a mouse ear. 15 hours after the injection, the ear was embedded in TissueTek O.C.T medium and 60 μm thick cryosections were prepared. The sections were stained overnight with X-gal. FIG. 12A shows cryosections of a mRNA transfer negative ear. No lacZ positive cells are detectable. FIG. 12B shows an overview. FIG. 12C shows a detailed view of a mRNA transfer positive ear. lacZ positive cells appear dark blue and are indicated by arrows.

FIG. 13 shows the compatibility of the Alexa Fluor 546 signal with the colour of the magenta-gal positive cells. In order to determine whether the detection of Alexa Fluor 546 in magenta-gal positive cells is possible, BHK cells were transfected with combinations of eGFP mRNA (eGFP=enhanced green fluorescence protein) or lacZ mRNA. The following combinations of transfected cells were analysed:
single transfections with only eGFP mRNA or lacZ mRNA
a mixture of such individually transfected cells (eGFP/lacZ) and
doubly transfected cells (eGFP+lacZ).

The cells were stained with an anti-eGFP antibody with Alexa Fluor 546 detection and subsequently with magenta-gal. Magenta-gal stained positive cells (which express lacZ) were detected by wide-field light microscopy (top row) and Alexa Fluor 546 stained positive cells (which express eGFP) were detected by fluorescence microscopy (middle row). The two results were superposed (bottom row) in order to obtain accurate results about the localisation of the cells relative to one another, although the Alexa 546 signal in this diagram covers the image of the light microscope. It is not possible to rule out that the direct uptake and self-translation of the supplied mRNA into the APCs takes place and is sufficient to trigger an immune response. In some APCs, processes of a slight or incomplete, undetectable translation might have taken place and (in the case of incomplete translation) might have effected the processing and presentation of the foreign antigen.

FIGS. 14 A-B show the specificity of MHC class II stainings of cryosections. 20 μg of mRNA coding for Escherichia coli β-galactosidase in a total volume of 100 μl of RL injection buffer with lactate were injected. 14 hours after the injection, the mice were sacrificed and the ears were removed. Transverse cryosections were prepared. The cryosections were first stained with an anti-MHC class II antibody (FIG. 14A) or the corresponding isotype control antibody (FIG. 14B) and detected by immunofluorescent staining with Alexa 546. The cryosections were then stained with magenta-gal (for β-galactosidase expression). The figures show magenta-gal stainings (left-hand column), MHC class II stainings (middle column, positions of lacZ positive cells are shown by outlining) and a superposition of both stainings (right-hand column, lacZ positive cells are indicated by outlining, MHC class II positive cells represent the light regions in the figure).

FIG. 15 shows the compatibility of cells which are X-gal dye and AEC dye positive. In order to determine whether the X-gal precipitate is compatible with the detection of AEC positive cells, BHK cells were co-transfected with eGFP mRNA and lacZ mRNA. The cells were stained with an anti-eGFP immune staining with AEC (red: positive cells, express eGFP), with a X-gal solution (blue-green: positive cells, express lacZ) or with a combination of AEC and X-gal. The stained cells were analysed by wide-light microscopy. Doubly positive cells appear black (black arrows). It is difficult to distinguish individually stained positive cells (green and red arrows) when the individual staining is strong and therefore tends to appear black.

FIGS. 16 A-C show the co-localisation of MHC class II positive and mRNA transfer positive cells. 20 μg of mRNA coding for β-galactosidase in a total volume of 100 μl of RL injection buffer with lactate were injected. 14 hours after the injection, the mice were sacrificed and the ears were removed. Transverse cryosections were prepared and were stained first with an anti-MHC class II antibody (FIG. 16A+B) or the corresponding isotype control antibody (FIG. 16C) (detected with Alexa 546 staining), then with X-gal (for βgalactosidase expression). Cells which are positive for the mRNA transfer appear green-blue, cells which are positive for MHC class II appear red, and doubly positive cells appear black. mRNA transfer positive cells are indicated by an arrow, independently of MHC class II expression.

FIG. 17 shows the mRNA transfer and the morphology of the ear muscle. 20 μg of mRNA coding for β-galactosidase in a total volume of 100 μl of RL injection buffer with lactate were injected. 14 hours after the injection, the mice were sacrificed and the ears were removed. Transverse cryosections were prepared and were stained first with X-gal (for β-galactosidase expression), then with haematoxylin and eosine. Cells which are positive for the mRNA transfer are indicated by arrows and are located close to the parenchyma cell layer.

### EXAMPLES

### Materials

1. Injection Buffers

The following buffers were used:
2× phosphate-buffered saline (PBS) (PBS 274 mM sodium chloride, 5.4 mM potassium chloride, 20 mM disodium hydrogen phosphate, 4 mM potassium dihydrogen phosphate, pH 7.3 at 20.8° C.),
2× HEPES-buffered saline (HBS) (HBS: 300 mM sodium chloride, 20 mM Hepes, pH 7.4 at 20.8° C.) and
1× RL injection buffer (without lactate) (82.2 mM sodium chloride, 4.3 mM potassium chloride, 1.44 mM calcium chloride, if no other composition and concentration has been indicated.
1× RL injection buffer with lactate (102.7 mM sodium chloride, 5.4 mM potassium chloride, 1.8 mM calcium chloride, 20 mM sodium lactate, if no other composition and concentration has been indicated.
1× RL injection buffer with lactate, without sodium chloride (4.3 mM potassium chloride, 1.44 mM calcium chloride, 22.4 mM sodium lactate, if no other composition and concentration has been indicated.
1× RL injection buffer with lactate, without potassium chloride (82.2 mM sodium chloride, 1.44 mM calcium chloride, 22.4 mM sodium lactate, if no other composition and concentration has been indicated.
1× RL injection buffer with lactate, without calcium chloride (82.2 mM sodium chloride, 4.3 mM potassium chloride, 22.4 mM sodium lactate, if no other composition and concentration has been indicated.

When 2× PBS and 2× HBS were used, all the components were dissolved in water and the pH was adjusted. Diethyl pyrocarbonate (DEPC, Sigma, Schnelldorf, Germany) was then added to a concentration of 0.1% (v/v). The buffers were incubated for over one hour at 37° C. The buffers were then autoclaved.

1× RL injection buffer with lactate was itself prepared from a 20× stock solution of the four different salts (sodium chloride, potassium chloride, calcium chloride and sodium lactate).

Likewise, the 1× RL injection buffer was prepared from a 20× stock solution of the three different salts (sodium chloride, potassium chloride and calcium chloride). In further experiments, sodium chloride or potassium chloride or calcium chloride was omitted without compensating for the lower osmolarity. These RL injection buffers with lactate, without NaCl, KCl or CaCl₂) were also prepared from a 20× stock solution. With the exception of the sodium lactate racemate solution (Fluka, Schnelldorf, Germany), each of these components was treated with DEPC and autoclaved, as described for 2× PBS and 2× HBS.

All the buffers and buffer components were checked for ribonuclease activity by incubating 1 μg of mRNA in 1× buffer for more than two hours at 37° C. In the analysis of the mRNA by means of formaldehyde-agarose gel electrophoresis, buffers in which no degradation was observed were used.

2. Mice

All animal experiments were carried out in accordance with institutional and national guidelines. Female BALB/c mice aged 8 to 15 weeks were obtained from Charles River (Sulzfeld, Germany).

Before the intradermal injection, the mice were anaesthetised and the ear muscle was treated with isopropanol. In order to analyse the mRNA uptake and translation, the mice were sacrificed after a specific time and the ears were removed and shaved with a razor blade in order to remove troublesome hairs.

3. Humans

Human experiments were carried out with healthy male volunteers, who had the background and possible consequences of the investigations explained to them and gave their consent.

### Example 1: Preparation of the Nucleic Acids

mRNA

“Capped” mRNA was prepared by means of in vitro “run-off” transcription with T7 RNA polymerase (T7-Opti mRNA kits, CureVac, Tubingen, Germany).

The coding sequence of this mRNA (either Escherichia coli β-galactosidase [lacZ] cloned from Acc. U02445, or Photinus pyralis luciferase [luc], cloned from Acc. U47295) was flanked at its 3′-ends by an alpha-globin untranslated region and an artificial poly A (n=70) tail. For the mouse experiments, the mRNA was extracted with phenol/chloroform/isoamyl alcohol and precipitated with lithium chloride. The mRNA was then resuspended in water and the yield was determined by spectrophotometry at 260 nm. Finally, the mRNA was precipitated with ammonium acetate and resuspended in a sterile manner in water.

pDNA

Endotoxin-free pCMV-luc DNA was prepared with the EndoFree Plasmid Maxi Kit (Qiagen, Hilden, Germany). The pDNA was precipitated with ammonium acetate and finally resuspended in a sterile manner in water. The pCMV-luc plasmid was modified by insertion of a Xba I— (blunted with Klenow fragment) Hind III fragment from pGL3 (Acc. U47295) into the Nsi I— (blunted with Klenow fragment) Hind III-digested plasmid of pCMV—HB—S(Acc. A44171). The reporter gene of the pDNA was under the control of the CMV promoter.

Stock Solutions

Stock solutions were prepared by diluting the mRNA or DNA in sterile water and determining the concentration and purity by spectrophotometry (at 260, 280 and 320 nm).

Quality Control

For all nucleic acid samples, the concentration was determined by spectrophotometry and the integrity was checked by means of formaldehyde-agarose gel electrophoresis (mRNA) or restriction digestion and TBE-agarose gel electrophoresis (DNA) (FIG. 10). In addition to the integrity, the translation capacity of all nucleic acid samples was analysed by electroporation of BHK21 cells. To this end, 1 to 3 million cells were electroporated in 200 μl of PBS with 10 μg of nucleic acid at 300 V and 150 μF in 0.4 cm cuvettes. The transfected cells were analysed for protein expression 8 to 24 hours after the electroporation, by a suitable detection method (X-gal staining or luminescence detection) (FIG. 10). For in vivo experiments, only nucleic acid samples that exhibited protein expression in BHK21 cells and suitable integrity in the gel electrophoresis were injected.

### Example 2: Preparation of the RNA Injection Solutions

For HBS and PBS, the mRNA was diluted in 1× concentrated buffer. For RL injection buffer with or without lactate and the individual variations of this (absence of one of the ions Ca²⁺, K⁺, Na⁺) (for compositions and concentrations see Materials, 1. Injection buffers), the mRNA was diluted in 0.8× concentrated buffer. Unless indicated otherwise, 20 μg of mRNA in 100 μl of injection buffer were used per mouse ear. In order to remove secondary structures in the mRNA, the RNA injection solutions were heated for 5 minutes at 80° C. Then the solutions were placed on ice for a further 5 minutes. Finally, the RNA injection solution was drawn into Sub-Q (Becton Dickinson, Heidelberg, Germany) injection syringes. Separate injection syringes were used for each injection. Plasmid DNA (pDNA) was diluted in 1× concentrated PBS.

### Example 3: Detection of Luciferase Activity Ex Vivo

In order to detect luciferase activity ex vivo, tissue lysates were prepared. To this end, the tissue was comminuted under liquid nitrogen using a pestle and mortar, and the remaining “lumps” were homogenised with 800 μl of lysis buffer (25 mM Tris HCl, 2 mM EDTA, 10% (w/v) glycerine, 1% (w/v) Triton X-100 plus freshly added 2 mM DTT and 1 mM PMSF). The supernatant of the homogenate was obtained after centrifugation (10 min, 13,000 rpm, 4° C.) in a minicentrifuge. 110 μl aliquots of this lysate were stored at −80° C.

In order to measure the luciferase activity, aliquots were thawed on ice and the light emission of 50 μl of lysate was measured for 15 seconds with a luminometer (LB 9507, Berthold, Bad Wildbad, Germany). The luminometer automatically added 300 μl of buffer A (25 mM glycyl glycine, 15 mM magnesium sulfate, 5 mM freshly added ATP, pH 7.8) and 100 μl of buffer B (250 μM luciferin in water) to the lysate before the measurement.

For standardisation, serial dilutions of recombinant luciferase protein (QuantiLum®, Promega, Madison, USA) were used in all the measurements. By means of this standard, the amount of luciferase molecules was calculated for each individual measurement. For each lysate, the luciferase activity was measured doubly on two different days and the mean value of the luciferase activity was calculated. The variation coefficient (n=4) for the amount of luciferase molecules was below 10% for all lysates having luciferase activity above the detection limit. This detection limit (indicated in all the diagrams by a thick line with a number) was calculated by means of the mean value of the measurements with only lysis buffer plus three times the standard deviation of these values (n=80).

### Example 4: In Vivo Bioluminescence Detection

In order to detect a luciferase injection in living animals, mice were anaesthetised at a specific time after the nucleic acid injection. The mice were divided into three different groups: in group I of mice, 100 μl of RL injection buffer were injected into the left ear and 20 μg of mRNA coding for luciferase in 100 μl of RL injection buffer were injected into the left ear. In group II, 20 μg of mRNA coding for luciferase in 100 μl of RL injection buffer were injected into each of the left and right ears. In group III of mice, 100 μl of RL injection buffer were injected into the right ear and 20 μg of mRNA coding for luciferase in 100 μl of RL injection buffer were injected into the left ear. The mice were then injected i.p. with 200 μl of 20 mg/ml luciferin (Synchem, Kassel, Germany) in PBS (sterile filtered). 5 minutes after the luciferin injection, the light emission of the mice was collected for a period of 20 minutes. To this end, the mice were positioned on a preheated plate (37° C.) in a darkened box (group I on the left, group II in the middle, group III on the right). The box was equipped with an Aequoria Macroscopic Imaging camera (Hamamatsu, Japan). The light emission was shown in a false-colour image, on which a greyscale image of the mouse is superposed. of the mice under normal light. The same experiment was carried out analogously using 20 μg of mRNA coding for luciferase in RL injection buffer with lactate or RL injection buffer with lactate, without sodium chloride, RL injection buffer with lactate, without potassium chloride, and RL injection buffer with lactate, without calcium chloride.

### Example 5: β-Galactosidase Activity and Histology

Shaved mouse ears were destroyed, embedded in medium containing Tissue-Tek® O.C.T™ compound (Sakura, Zoeterwuode, Netherlands) and stored at −80° C. From these blocks, 20 successive 20 μm thick transverse cryosections were placed in 5 sets (FIG. 11) on SuperFrost® plus specimen holders (Langenbrinck, Emmendingen, Germany) in such a manner that the vertical distance between two sections of a set was approximately 100 μm. The sections were then dried in air and stored at −20° C. until they were stained. For a first screening as to the area in which the transferred mRNA (coding for Escherichia coli βgalactosidase) has been taken up and translated, 1 set of sections were stained with X-gal. To this end, the specimen holders were exposed to room temperature and outlined with a ImmEdge™ pen (Vektor, Burlingame, USA). Then the sections were fixed for 15 minutes with 2% formalin in PBS. The specimen holders were then washed 3× for 2 minutes with PBS and then stained overnight at 37° C. in a humidity chamber with X-gal staining solution (1 mg/ml freshly added X-gal, 5 mM potassium ferricyanide, 5 mM potassium ferrocyanide, 1 mM magnesium chloride, 15 mM sodium chloride, 60 mM disodium hydrogen phosphate, 40 mM sodium dihydrogen phosphate). The staining was terminated by washing the specimen holders 2× for 2 minutes and treating them with Hydro-Matrix® (Micro-Tech-Lab, Graz, Austria, diluted twice in water) medium.

In order to obtain information about the tissue morphology, the X-gal staining was combined with a haematoxylin-eosine (HE) staining for another set of sections. To this end, after the X-gal staining, the sections were washed 3× for 2 minutes in PBS and additionally for 5 minutes in bidistilled water, before a 2-second staining with Mayers haemalaun (Merck, Darmstadt, Germany) was carried out. The staining was developed for 10 min under running tap water, before counter-staining was carried out for 10 min with 0.1% eosine Y (Sigma, Schnelldorf, Germany) in water. The staining was stopped by washing briefly in bidistilled water, followed by dehydration with increasing alcohol concentrations (2 minutes 80% ethanol, 2 minutes 95% ethanol, 2 minutes 100% ethanol, 5 minutes 100% xylene). Finally, the dried sections were treated with Roti®-Histokitt (Roth, Karlsruhe, Germany) medium.

In order to determine whether the target cells transfected with the mRNA are antigen-presenting cells, a double staining for MHC class II molecules (expressed by APC) and mRNA transfer (relating to β-galactosidase expression) was carried out. Both immunohistochemical and immunofluorescent detection of the MHC class II molecules were carried out. For both protocols, the sections were washed between all three steps 3× for 2 minutes with PBS. For the immunohistochemical procedure, the sections were fixed with 1% (w/v) formalin (Fluka) in PBS. The lipids were then removed by incubation for 30 seconds in pure acetone. Immediately thereafter, blocking was carried out for 30 minutes at room temperature with 4% goat's blood (Vektor Laborotories Inc., Burlingame, Calif.) and 50 μg/ml avidin D (Vektor Laboratories Inc., Burlingame, Calif.) in PBS. The remaining biotin binding sites were blocked with 50 μg/ml biotin (AppliChem, Darmstadt, Germany) and at the same time stained for MHC class II molecules with the monoclonal antibody 2G9 (Becton Dickinson, Heidelberg, Germany) or the suitable isotype control antibody (rat IgG 2a, R35-95, Becton Dickinson, Heidelberg, Germany), in each case diluted to 1 μg/ml (all in PBS). Thereafter, the sections were incubated for 30 minutes at room temperature with biotinylated goat/anti-rat IgG (3 μg/ml) vector and 2% mouse serum (CCPro, Neustadt, Germany) in PBS. ABC complex (1:100 of reagent A and B in PBS (Vektor Laboratories Inc., Burlingame, Calif.) was then added for 30 minutes at room temperature. The MHC class II staining was completed by detection with freshly prepared 3-amino-9-ethylcarbazole (AEC, Sigma) substrate solution (0.5 mg/ml AEC, 0.015% hydrogen peroxide, 50 mM sodium acetate, pH 5.5) which had been filtered through a 0.45 μm filter. The substrate reaction was stopped by washing twice for 5 minutes with water and washing three times for 5 minutes with PBS. An X-gel staining was then carried out, as described above.

A similar staining protocol was used for the immunofluorescent detection. Following the acetone step, the sections were blocked for 50 minutes at room temperature in blocking buffer (1% bovine serum albumin in PBS). The sections were then incubated for 40 minutes with primary antibodies (2G9 or isotype control antibodies), diluted to 1 μg/ml in blocking buffer. Incubation was then carried out for 40 minutes at room temperature with Alexa Fluor 546 goat/anti-rat IgG (1:400; Molecular Probes, Leiden, Netherlands) in blocking buffer. Finally, a magenta-gal staining was carried out. To this end, X-gal in the staining solution was replaced with 0.1 mg/ml magenta-gal (Peqlab, Erlangen, Germany).

The sections were analysed with a Zeiss (Oberkochen, Germany) Axioplan 2 microscope equipped with an Axiocam HRc camera and Axiovision 4.0 software. Colours and contrast in the photographs were adjusted linearly.

### Example 6: RNA Transfer and Translation in Humans

This experiment was carried out with healthy male volunteers. The injection sites were shaved, disinfected and treated with RnaseZap (Ambion, Austin, USA) solution. Then 120 μg of mRNA in 0.8× RL injection buffer was injected in a single batch in a total volume of 200 μl. In further analogous batches there was used instead of RL injection buffer:
RL injection buffer with lactate, without sodium chloride,
RL injection buffer with lactate, without potassium chloride and
RL injection buffer with lactate, without calcium chloride.

15 hours after the injection, biopsies having a diameter of 4 mm were taken (stamped out) under local anaesthetic. The biopsies were shock-frozen in liquid nitrogen and prepared as described (Example 3). The ground biopsies were resuspended in 600 μl of lysis buffer.

Statistic Evaluation

The mean values of two different groups were compared by the so-called “non-parametric Mann-Whitney rank sum test”. A p value of <0.05 was regarded as a significant difference and shown in the diagrams.

### Example 7: Comments on the Various Staining Processes Carried Out

In order to identify the cell type which takes up and expresses the mRNA in vivo, a histological process was used, which permits the detection of the mRNA transfer in conjunction with a cell-type-specific staining.

Because the mRNA transfer could not be detected with fluorescent probes, a process was carried out in which mRNA coding for Escherichia coli 3-galactosidase was used in combination with various indigo dyes (X-gal or magenta-gal).

1. Special Features of the 3-Galactosidase Detection System

In order to ensure that all the cells were present in a single layer for the indigo staining process, thin sections of the mouse ear were prepared. Taking into account the morphology of the ear muscle of the mice (a thin layer of a thickness of approximately from 0.5 to 1 mm) and the fact that only cryosections can be used β-galactosidase is heat-inactivated during some steps which are necessary in order to prepare paraffin sections) and the requirement of sections and as many sub-sections as possible of high quality, the preparation of these sections proved to be very difficult. Nevertheless, it was possible to prepare several sets of sections of good quality having a thickness of 20 μm. Two different dyes were used to detect the β-galactosidse activity. For X-gal (positive cells were stained blue-green), results were achieved with a better contrast than with magenta-gal (positive cells were stained violet). At the same time, however, unspecific background stainings, for example caused by hair follicles, were very visible in the X-gal staining. Nevertheless, a clear distinction of the mRNA transfer was possible. An unspecific background staining was not visible for magenta-gal.

2. Requirements for Combined Indigo and Immune Staining

The combination of mRNA transfer staining (indigo dye) and cell-specific marker staining (specific antibodies) required several adaptations regarding the fixing agent and the sequence of the combined stainings (indigo staining included 14-hour incubation at 37° C.). The best results for the antibody staining (against MHC II molecules) were obtained when first acetone fixing and the antibody staining were carried out. By contrast, the best results for βgalactosidase activity were achieved when first fixing with a mixture of formaldehyde and glutardialdehyde and the indigo staining were carried out. Taking into account these various circumstances, the following process was chosen: fixing with formaldehyde, but without glutardialdehyde and the antibody staining. Glutaraldehyde had to be omitted because it drastically increases the autofluorescence of the tissue, while it had only a slight, if any, effect on the quality of the indigo staining. Fixing with formaldehyde was used for several reasons:
1. the morphology of the tissue was better protected thereby than with acetone,
2. a sharp and strong indigo staining requires fixing with formaldehyde, and
3. the quality of the anti-MHC II antibody staining was nevertheless acceptable with formaldehyde.

A short incubation was then carried out in pure acetone in order to remove lipids and fats. This permitted better quality (fewer or no air bubbles) with the water-soluble medium used. Finally, antibody stainings of good quality were only achieved when this staining was carried out first. The staining sequence (formaldehyde fixing) had only a slight effect on the quality of the indigo staining.

3. Dye Compatibility in Combined Indigo and Immune Stainings

The combination of two different stainings required not only the compatibility of the various steps of the two protocols but also the compatibility of the probes used for the detection. In principle, an immune staining is possible with precipitating dyes (enzymatic probe) or with fluorescent dyes (labelled probe). In order to combine the indigo staining with a precipitating dye, X-gal and AEC were used. Doubly positive cells appeared black in such a staining (FIG. 158). It can be difficult to distinguish intensely stained individual positive cells. The combination of magenta-gal with the fluorescent dye Alexa Fluor 546 was therefore preferred. The use of magenta-gal instead of X-gal was preferred for two reasons:
1. the intensity of the magenta-gal staining was much weaker (even when the dye was added in saturated amounts),
2. the colour of magenta-gal positive cells corresponded better with the emission wavelength of the Alexa Fluor 546 fluorescent dye.

Both factors minimise quenching of the Alexa Fluor 546 fluorescent signal. This dye combination actually permitted the detection of both signals (FIG. 13), at least when the indigo staining was not too strong (this was the case for the mRNA transfer positive cells in the sections).

### LIST OF REFERENCES

1. Ulmer J. B., Curr. Opin. Drug Discov. Devel., 2001, 4:192-197
2. J. A. Wolff et al., Science 247, 1465-1468 (1990).
3. H. L. Robinson, L. A. Hunt, R. G. Webster, Vaccine 11, 957-960 (1993).
4. J. B. Ulmer et al., Science 259, 1745-1749 (1993).
5. J. A. Wolff et al., Science 247, 1465-1468 (1990).
6. D. Boczkowski, S. K. Nair, D. Snyder, E. Gilboa, J.Exp.Med. 184, 465-472 (1996).
7. J. P. Carralot et al., Cell Mol.Life Sci. (2004).
8. R. M. Conry et al., Cancer Res. 55, 1397-1400 (1995).
9. R. D. Granstein, W. Ding, H. Ozawa, J Invest Dermatol 114, 632-636 (2000).
10. I. Hoerr, R. Obst, H. G. Rammensee, G. Jung, Eur. J Immunol. 30, 1-7 (2000).
11. J. Donnelly, K. Berry, J. B. Ulmer, Int J Parasitol. 33, 457-467 (2003).
12. D. M. Klinman et al., Springer Semin.Immunopathol. 19, 245-256 (1997).
13. P. Forg, P. von Hoegen, W. Dalemans, V. Schirrmacher, Gene Ther. 5, 789-797 (1998).

1-21. (canceled)

22. A method for increasing the expression of a selected antigen in an organism comprising:
a) obtaining an aqueous mRNA injection solution comprising a calcium salt and a purified mRNA encoding the selected antigen, wherein said mRNA comprises (i) a 5′ cap; (ii) a poly-A tail of at least 25 nucleotides; and (iii) a 5′ and/or 3′ untranslated region (UTR); and
b) injecting the aqueous mRNA injection solution into the organism intradermally or intranodally,
thereby expressing the selected antigen in the organism.

23. The method of claim 22, wherein the aqueous mRNA injection solution comprises salts with the anions selected from the group consisting of chlorides, iodides, bromides, hydroxides, carbonates, hydrogen carbonates, and sulfates.

24. The method of claim 22, wherein the aqueous mRNA injection solution contains at least 50 mM sodium chloride (NaCl), at least 0.01 mM calcium chloride (CaCl₂), and optionally at least 3 mM potassium chloride (KCl).

25. The method of claim 24, wherein the aqueous mRNA injection solution contains from 50 mM to 800 mM sodium chloride (NaCl), from 0.01 mM to 100 mM calcium chloride (CaCl₂), and optionally from 3 mM to 500 mM potassium chloride (KCl).

26. The method of claim 22, wherein the aqueous mRNA injection solution further comprises lactate.

27. The method of claim 26, wherein the aqueous mRNA injection solution contains at least 50 mM sodium chloride (NaCl), at least 0.01 mM calcium chloride (CaCl₂), at least 15 mM lactate, and optionally at least 3 mM potassium chloride (KCl).

28. The method of claim 26, wherein the aqueous mRNA injection solution contains from 15 mM to 500 mM lactate.

29. The method of claim 22, wherein the mRNA is complexed with a polycationic polypeptide.

30. The method of claim 29, wherein the polycationic polypeptide is poly-L-lysine, poly-L-arginine, or protamine.

31. The method of claim 30, wherein the polycationic polypeptide is protamine.

32. The method of claim 22, wherein the mRNA is a modified mRNA comprising at least one modification selected from the group consisting of: a) having an increased G/C content in the coding region of the mRNA relative to the wild-type (WT) mRNA, b) having at least one analog of a naturally occurring ribonucleotide, c) being free of one or more destabilizing sequences in the WT sequence, d) having an increased A/U content in the ribosome binding site of the mRNA relative to the WT mRNA, e) having one or more codon substitutions, f) having a heterologous poly(A) tail, g) having an IRES sequence, h) having a 3′ or 5′ stabilization sequence, and i) having a sequence encoding a signal peptide.

33. The method of claim 22, wherein the mRNA does not comprise a chemical modification.

34. The method of claim 22, wherein the mRNA encodes a tumor antigen.

35. The method of claim 34, wherein the tumor antigen is selected from the group consisting of MAGE, NY-ESO-1, PSA, and mucin.

36. The method of claim 22, wherein the aqueous mRNA injection solution is a Ringer's or a Ringer's Lactate solution.

37. The method of claim 22, wherein the aqueous mRNA injection solution does not contain mono-, di- or polysaccharides.

38. The method of claim 22, wherein the aqueous mRNA injection solution comprises from 0.5 mM to 100 mM calcium salt.

39. The method of claim 38, wherein the aqueous mRNA injection solution comprises from 1.5 mM to 40 mM calcium salt.

40. The method of claim 22, comprising injecting the aqueous mRNA injection solution into the organism intranodally.

41. The method of claim 22, wherein the organism suffers from a disease or disorder selected from cancer, allergies, autoimmune diseases, viral infection, and bacterial infection.

42. A method for eliciting an immune response in an organism comprising injecting an aqueous mRNA injection solution into an organism intradermally or intranodally, wherein the aqueous mRNA injection solution comprises a calcium salt and a purified mRNA comprising: (i) a 5′ cap; (ii) a poly-A tail of at least 25 nucleotides; and (iii) a sequence encoding a selected antigen, thereby eliciting an immune response against the selected antigen within the organism.

43. An injection syringe comprising an aqueous mRNA injection solution comprising a calcium salt and a purified mRNA comprising: (i) a 5′ cap; (ii) a poly-A tail of at least 25 nucleotides; and (iii) a sequence encoding a selected antigen, said aqueous mRNA injection solution comprised in the injection syringe.


SEQUENCE LISTING (34 sequences)

SEQ ID NO: 1 - RNA, 13 nt, Unknown
  Kozak sequence (see description page 16)
gccgccaccaugg

SEQ ID NO: 2 - RNA, 15 nt, Unknown
  Stabilising sequence of the general formula (C/U)CCANxCCC(U/A)PyxUC(C/U)CC (see description page 18)
  misc_feature (1)..(1): n = c or u
  misc_feature (5)..(5): n = a, c, u, g or others
  repeat_unit (5)..(5): x = as desired
  misc_feature (9)..(9): n = u or a
  repeat_unit (10)..(10): x = as desired
  misc_feature (10)..(10): n = Py (pyrimidine)
  misc_feature (13)..(13): n = c or u
nccancccnnucncc

SEQ ID NO: 3 - RNA, 75 nt, Artificial
  Description of the sequence signal sequence HLA-B*07022
  CDS (1)..(75)
augcuggugauggccccgcggaccguccuccugcugcugagcgcggcccuggcccugacggagaccugggccggc

SEQ ID NO: 4 - protein, 25 aa, Artificial
  Synthetic Construct
MLVMAPRTVLLLLSAALALTETWAG

SEQ ID NO: 5 - RNA, 75 nt, Artificial
  Description of the sequence signal sequence HLA-A*3202
  CDS (1)..(75)
auggccgugauggcgccgcggacccugcuccugcugcugcugggcgcccuggcccucacgcagaccugggccggg

SEQ ID NO: 6 - protein, 25 aa, Artificial
  Synthetic Construct
MAVMAPRTLLLLLLGALALTQTWAG

SEQ ID NO: 7 - RNA, 75 nt, Artificial
  Description of the sequence signal sequence HLA-A*01011
  CDS (1)..(75)
auggccgugauggcgccgcggacccugcuccugcugcugagcggcgcccuggcccugacgcagaccugggccggg

SEQ ID NO: 8 - protein, 25 aa, Artificial
  Synthetic Construct
MAVMAPRTLLLLLSGALALTQTWAG

SEQ ID NO: 9 - RNA, 75 nt, Artificial; identical to SEQ ID NO: 7
  Description of the sequence signal sequence HLA-A*0102
  CDS (1)..(75)

SEQ ID NO: 10 - protein, 25 aa, Artificial
  Synthetic Construct
MAVMAPRTLLLLLSGALALTQTWAG

SEQ ID NO: 11 - RNA, 75 nt, Artificial
  Description of the sequence signal sequence HLA-A*0201
  CDS (1)..(75)
auggccgugauggcgccgcggacccugguccuccugcugagcggcgcccuggcccugacgcagaccugggccggg

SEQ ID NO: 12 - protein, 25 aa, Artificial
  Synthetic Construct
MAVMAPRTLVLLLSGALALTQTWAG

SEQ ID NO: 13 - RNA, 75 nt, Artificial; identical to SEQ ID NO: 7
  Description of the sequence signal sequence HLA-A*0301
  CDS (1)..(75)

SEQ ID NO: 14 - protein, 25 aa, Artificial
  Synthetic Construct
MAVMAPRTLLLLLSGALALTQTWAG

SEQ ID NO: 15 - RNA, 75 nt, Artificial; identical to SEQ ID NO: 7
  Description of the sequence signal sequence HLA-A*1101
  CDS (1)..(75)

SEQ ID NO: 16 - protein, 25 aa, Artificial
  Synthetic Construct
MAVMAPRTLLLLLSGALALTQTWAG

SEQ ID NO: 17 - RNA, 75 nt, Artificial; identical to SEQ ID NO: 3
  Description of the sequence signal sequence HLA-B*070201
  CDS (1)..(75)

SEQ ID NO: 18 - protein, 25 aa, Artificial
  Synthetic Construct
MLVMAPRTVLLLLSAALALTETWAG

SEQ ID NO: 19 - RNA, 75 nt, Artificial
  Description of the sequence signal sequence HLA-B*2702
  CDS (1)..(75)
augcgggugaccgccccgcgcacgcugcuccugcugcuguggggcgcggucgcccugaccgagaccugggccggg

SEQ ID NO: 20 - protein, 25 aa, Artificial
  Synthetic Construct
MRVTAPRTLLLLLWGAVALTETWAG

SEQ ID NO: 21 - RNA, 78 nt, Artificial
  Description of the sequence signal sequence HLA-Cw*010201
  CDS (1)..(78)
augcgggugauggccccgcgcacccugauccuccugcugagcggcgcgcuggcccugacggagaccugggccugcucg

SEQ ID NO: 22 - protein, 26 aa, Artificial
  Synthetic Construct
MRVMAPRTLILLLSGALALTETWACS

SEQ ID NO: 23 - RNA, 78 nt, Artificial
  Description of the sequence signal sequence HLA-Cw*02021
  CDS (1)..(78)
augcgggugauggccccgcgcacccugcuccugcugcugagcggcgcgcuggcccugacggagaccugggccugcucg

SEQ ID NO: 24 - protein, 26 aa, Artificial
  Synthetic Construct
MRVMAPRTLLLLLSGALALTETWACS

SEQ ID NO: 25 - RNA, 75 nt, Artificial
  Description of the sequence signal sequence HLA-E*0101
  CDS (1)..(75)
augguggacggcacccugcuccugcugagcucggaggcccuggcgcugacgcagaccugggccgggagccacagc

SEQ ID NO: 26 - protein, 25 aa, Artificial
  Synthetic Construct
MVDGTLLLLSSEALALTQTWAGSHS

SEQ ID NO: 27 - RNA, 87 nt, Artificial
  Description of the sequence signal sequence HLA-DRB1
  CDS (1)..(87)
auggugugccugaagcucccgggcgggagcugcaugaccgcccugacggucacccugauggugcugucgagcccccuggcgcuggcc

SEQ ID NO: 28 - protein, 29 aa, Artificial
  Synthetic Construct
MVCLKLPGGSCMTALTVTLMVLSSPLALA

SEQ ID NO: 29 - RNA, 75 nt, Artificial
  Description of the sequence signal sequence HLA-DRA1
  CDS (1)..(75)
auggccaucagcggcgugccgguccugggguucuucaucaucgcggugcucaugucggcccaggagagcugggcc

SEQ ID NO: 30 - protein, 25 aa, Artificial
  Synthetic Construct
MAISGVPVLGFFIIAVLMSAQESWA

SEQ ID NO: 31 - RNA, 87 nt, Artificial
  Description of the sequence signal sequence HLA-DR4
  CDS (1)..(87)
auggugugccugaaguucccgggcgggagcugcauggccgcgcucaccgucacgcugauggugcugucgagcccccuggcccuggcc

SEQ ID NO: 32 - protein, 29 aa, Artificial
  Synthetic Construct
MVCLKFPGGSCMAALTVTLMVLSSPLALA

SEQ ID NO: 33 - RNA, 87 nt, Artificial
  Myelin oligodendrocyte glycoprotein ( see description page 21)
  CDS (1)..(87)
auggccugccuguggagcuucucguggccgagcugcuuccucagccugcugcugcugcugcuccugcagcugagcugcagcuacgcg

SEQ ID NO: 34 - protein, 29 aa, Artificial
  Synthetic Construct
MACLWSFSWPSCFLSLLLLLLLQLSCSYA